(19) United States
(12) Patent Application Publication
(10) Pub. No.: US 2022/0000289 A1
(43) Pub. Date: Jan. 6, 2022

(54) ADJUSTABLE INFLATABLE NECK SUPPORT DEVICE AND METHOD FOR MANUFACTURING THE SAME

(71) Applicant: Hoi Ming Michael HO, Tuen Mun (HK)

(72) Inventor: Hoi Ming Michael HO, Tuen Mun (HK)

(21) Appl. No.: 17/343,952

(22) Filed: Jun. 10, 2021

Related U.S. Application Data
(63) Continuation-in-part of application No. 17/147,448, filed on Jan. 12, 2021.

(30) Foreign Application Priority Data
Jul. 3, 2020 (CN) 202010631822.5
Dec. 14, 2020 (CN) 202011467755.4
Jun. 4, 2021 (CN) 202110625030.1

(51) Int. Cl. A47G 9/10; A61H 1/02; A61H 9/00; A61N 1/36

(52) U.S. Cl. CPC A47G 9/1027; A47G 9/109; A47G 9/1036; A61H 1/0296; A61H 9/0078; A61H 2203/0475; A61H 2205/04; A61H 2201/10; A61H 2201/0207; A61H 2203/0456; A61N 1/36021

(57) ABSTRACT

An adjustable inflatable neck support device includes a supporting portion and a bearing portion whose height is smaller than that of the supporting portion. The supporting portion has a center support body and can be provided therein with a first inflatable airbag. Two second inflatable airbags can be provided in the neck support device. An inflation device can be connected to the airbags. When a user rests on the neck support device in a supine position or on the side of the head, the top surface thereof can be adjusted to support the neck. The inflation device inflates or deflates the airbags to displace the top surface of the neck support device to change the force exerted on the neck by the center support body and/or areas of the top surface of the neck support device that correspond in position to the airbags to restore and maintain head/spine alignment.

### CROSS-REFERENCE TO RELATED PATENT APPLICATION

This non-provisional application is a continuation-in-part application of co-pending U.S. application Ser. No. 17/147,448, filed on Jan. 12, 2021, which claims priority to and the benefit of under 35 U.S.C. § 119(a), Chinese Patent Application No. 202011467755.4, filed on Dec. 14, 2020 in the People's Republic of China, and Chinese Patent Application No. 202010631822.5, filed on Jul. 3, 2020 in the People's Republic of China. The entire content of each and every of the above identified applications is incorporated herein by reference.

This non-provisional application also claims priority to and the benefit of, under 35 U.S.C. § 119(a), Chinese Patent Application No. 202110625030.1, filed on Jun. 4, 2021 in the People's Republic of China. The entire content of the above identified application is incorporated herein by reference.

### FIELD

The present disclosure relates to a neck support device, and more particularly to a neck support device provided with three independent inflatable airbags that are adjustable therein, so that a user can inflate or deflate the inflatable airbags by himself or herself, thereby changing the appearance, size, height and firmness of the neck support device, and adjusting the force exerted by the neck support device on the neck and head of the user, so as to afford better neck and head support and to help restore and maintain the neutral anatomical spinal alignment of the head, cervical spine and upper thoracic spine for both back sleepers and side sleepers. The neck support device according to the present disclosure is easy to use by either a back steeper or a side sleeper, resembles a comfortable pillow, and a user can use the neck support device while he or she is laying down or is sleeping.

### BACKGROUND

Conventional pillow designs involve merely manufacturing a filling bag, and filling therein soft and fluffy materials such as cotton balls, a shaped foam, foam chips, foam strips or other airy fluffy materials. While conventional pillows may have certain softness and repositionability at beginning, with the prolonged use time, the fluffiness of the pillows gradually decreases, and their supportability is therefore tampered. With pillows that are made with soft fluffy materials, after being pressed by the head and neck for a long time, these pillows become misshaped, and the head angle of a user when the user rests his or her head thereon would be too low or too high, which results in excessive neck muscle fatigue and strain, causing pain in the neck and shoulders, cervicogenic headaches and affects the smoothness of breathing. Moreover, the soft and fluffy materials in conventional pillows can also become deformed and tangled into blocks due to long-term compression, resulting in poor air permeability. Pillows with poor air permeability can have restricted air flow in the pillows, and therefore cause health problems to a user who is used to sleep in a prone position or on his or her sides, and can even cause more serious safety problems to a user suffering from sleep apnea. Further, a conventional pillow does not accommodate for different body contour and sleeping habits of different users, and its height cannot be adjusted to suit the height of the neck and head of a user, so as to provide proper support to, and restore and maintain the neutral anatomical spinal alignment of, the head, cervical spine and upper thoracic spine of the user. More specifically, take molded shaped pillows, otherwise known as orthopedic pillows for example, the types of foam used in a molded shaped pillow can be either too soft or too firm. A molded shaped pillow made with high density rebound foam can be very rigid and firm, and if its shape does not fit the shape of a user, the user can suffer from neck pain and discomfort. On the other hand, even though a shaped pillow made with softer memory foam may produce better conformity to the shape of neck and head of a user as compared to a pillow made with high density rebound foam, it tends to be too soft and does not rebound to the original molded shaped. Moreover, it becomes flat during use, and therefore does not fit the shape of the head and neck of the user. Furthermore, as the materials inside a traditional pillow are consistent throughout the pillow, the softness or firmness across the entire surface of such a pillow is the same, and does not vary to meet the different requirement for the softness/firmness of different body parts of a user, such as that of the head and of the neck. In summary, the lack of support for the head and neck of a user causes pain and discomfort to the user. If the foam density of a pillow is too high or too low, the pillow becomes uncomfortable and in turn causes pain to the user. The shape mismatching between that of the molded foam pillow and that of the neck and head of a user also leads to discomfort and pain.

The severeness of the afore-referenced issues only becomes more obvious given the fact that cervical vertebrae connect the skull on the superior side and the thoracic spine on the inferior side, and their importance is self-evident. However, people in modern time are suffering from cervical spondylosis (also referred to as cervical spine syndromes) more because of whiplash injuries from accidents, sports related injuries or fall, and more prevalently from inappropriate life style habits (such as prolonged use of mobile phones, game consoles or computers with the head lowered down, or improper standing or sitting posture), work needs (such as driving) that keep the neck at a fixed posture for a long time, or excessive psychological pressure, etc. These factors cause a user's neck muscles to become increasingly tense over time, the muscles to become rigid and shortened over time, and bad postural malalignment of the cervical spine, which even leads to cervical spine joint degeneration, or intervertebral discs degeneration and herniation (as they undertake excessive compression), leading to greater proneness to nerve impingement, arthritis, joint pain, and premature degeneration or thinning of intervertebral discs, etc., and eventually the development of symptoms such as throbbing arm pain, arm numbness, chronic headache, chronic fatigue, insomnia and chronic neck and shoulder pain, etc.

Most cervical spondylosis, especially chronic neck and shoulder dysfunction, can be caused by conditions such as tightness of the neck and shoulder muscles or excessive compression of the cervical spine joints that cause nerve irritation and impingements. Such a patient can suffer from neck pain almost every day, and the pain can become worse especially after a whole day of work, making it difficult for the patient to relax and fall asleep at night. In addition, an ordinary pillow available on the market provide little, if not none, benefit to a patient with cervical spondylosis; rather, after resting on such a pillow for a long time, the patient can suffer from more severe neck and shoulder pain, headache and/or arm pain or numbness.

Indeed, most patients often cannot afford the time or money required for the conventional treatment, resulting in such patients not receiving treatment and suffering pain that could have been alleviated. In addition, many of the patients receiving conventional treatments have not been attended by proper medical professional capable of providing appropriate treatment the patients need. Therefore, such patients' pain has not been effectively relieved. In the case of having received no treatment or ineffective treatment, some patients try to take painkillers, use ointments or patches, etc. However, their effects are limited, and long-term use of such can have serious side effects.

Since people's head and neck are unique in size, shape, length and flexibility, it is extremely difficult to create a conventional filled pillow or molded shaped pillow that can fit and support everyone's head and neck properly. Further, as people tend to move during sleep, a traditional filled pillow or a molded shaped pillow cannot fit and support the head and neck of a user equally well when he or she is sleeping in the supine position and when they sleep on their right and left sides. That is, a one size pillow with a fixed density or shape simply cannot fit and support the head and neck of a user equally well for both side and back sleep posture. Furthermore, sleeping on a pillow that does not help to support and maintain the natural neutral postural spinal alignment of the head, neck and upper back of a user can cause him or her spinal misalignment, straining of paraspinal muscles, irritation and pinching of the nerves, which can in turn lead to acute and chronic neck and shoulder pain, cervicogenic headaches and numbing pain in the upper limbs. The discomfort and pain in the neck and shoulder further cause such a user trouble falling asleep or being able to sleep deeply.

That is, many people, either back sleepers or side sleepers, lack proper support to their cervical spine, and cannot restore and maintain the neutral anatomical spinal alignment of the head, cervical spine and upper thoracic spine when they rest or sleep, and therefore have suffered from chronic headaches, neck and shoulder dysfunction and the resulting pain due to neck muscles tightness, excessive compression of cervical spine joints, herniated intervertebral discs, impingement plus irritation of paraspinal nerves, etc., and therefore from deteriorated health condition and life quality, without a good solution being availed. Accordingly, effectively solving the aforementioned issues, so that either a back sleeper or a side sleeper can have his or her neck contour and sleeping habits suited by a matching neck support device with increased sleep or rest comfort, and does not need to spend huge amount of time and/or money on cervical treatment, but can independently alleviate and/or prevent cervical syndromes by having proper and adjustable neck support, has become an important subject of the present disclosure.

### SUMMARY

One aspect of the present disclosure is directed to a neck support device that can be shaped as a pillow and be embedded with three adjustable air cushions, for example, inflatable airbags, within three separate hollow chambers inside the neck support device. In certain embodiments, the neck support device can be made of molded composite foam. The combination of the adjustable inflatable airbags and the material of which the neck support device is made can work to lift the head and/or face of a user, either a back sleeper or a side sleeper, upwards until his or her head, cervical spine and upper thoracic spine becomes aligned in a straight line. This adjustable inflatable neck support device enables a user to adjust the neck support device to keep his or her head, neck and upper back in their natural neutral anatomical alignment, to prevent strain and pain whether he or she is laying or sleeping on the sides or in a supine position.

In certain embodiments, the neck support device includes from the front to the rear a supporting portion including a center support body and a first inflatable airbag and a bearing portion. The bearing portion can bear an occiput of a user when the user is in a supine position. A height of a top surface of the bearing portion is lower than a height of a top surface of the supporting portion. The center support body is located at a center portion of the supporting portion. The center support body has a top surface configured to support the neck of a user, and a bottom surface concavely provided with a first chamber. The first inflatable airbag can be accommodated within the first chamber, expand or contract along a displacement axis, and displace a top surface of the center support body along the displacement axis. The neck support device further includes two second inflatable airbags and an inflation device. Each of the two second inflatable airbags can be accommodated within a corresponding one of second chambers that are concavely provided on a bottom surface of the neck support device and located at opposite sides of the first chamber, expand or contract along the displacement axis, and displace a top surface of the neck support device along the axis. The inflation device can be connected to the first inflatable airbag and the second inflatable airbags, respectively, and inflate or deflate the first inflatable airbag and the second inflatable airbags together, or separately, to change expansion or contraction degrees of the first inflatable airbag and the second inflatable airbags. Furthermore, the inflation device can be either a manually operated, or a powered inflation device. Therefore, a user can operate the inflation device to adjust the expansion or contraction degrees of the first inflatable airbag and the second inflatable airbags by himself or herself, so as to change the force exerted on the neck by the center support body and/or areas of the top surface of the neck support device that correspond in position to the inflatable airbags to restore and maintain the neutral anatomical spinal alignment of the head, cervical spine and upper thoracic spine.

In certain embodiments, the inflation device includes a plurality of pipe bodies, a first air valve that can be assembled to one of the pipe bodies, at least one second air valve that can be assembled to another one of the pipe bodies, and at least one inflation portion. The inflation portion can be connected to the first inflatable airbag and the second inflatable airbags through the pipe bodies, inflate or deflate the first inflatable airbag when the first air valve is opened, and inflate or deflate the second inflatable airbags when the second air valve is opened.

In certain embodiments, the first and second chambers are specifically sized to allow the first and second inflatable airbags adequate amount of space needed for various level of expansions as desired by a user when inflating the airbags, so that the user can customize the level of firmness/softness and height of the neck support device according to the user's unique requirements. At least one of the airbags can be designed with an expandable top end and an non-expandable or minimal-expandable base, so that the majority of the expansion of the airbags when inflated can occur at the upper side of the airbags and in a direction towards the user resting on the neck support device, and expansion downwards towards the bottom of the neck support device can be minimized, leading to the airbags pushing upwards towards the neck and head of the user when inflated, as downward expansion of the air cushions can cause excessive expansion and outward forces against the neck support device structure and in turn damages thereto. That is, such a minimal flexible/expandable base of an airbag helps to prevent potential damage to the structure of the neck support device while directing the expansion upwards towards the user.

In certain embodiments, the first inflatable airbag that can be located in the first chamber is semi-tubular in shape, and has a rounded first end that is to face an upper inner surface of the first chamber, and an opposite second end that is a flat base and is to face a bottom opening of the first chamber. When inflated, for example, by a user actively pumping air into the first inflatable airbag, the first inflatable airbag rises upwards towards the posterior side of the cervical spine of the user who is laying on the neck support device in a supine position, and can match the anatomical shape of his or her cervical spine. When the user inflates the airbag to a level of height and softness/firmness that provides him or her with the desired customization, with the combined effect provided by the inflated airbag and the material of the neck support device that is holding the airbag, for example, orthopedic shaped memory foam, the neck support device can conform comfortably against the slightly lordotic curvature of the cervical spine of the user in a supine position, and provide customized support for his or her head and neck, so that the head, neck and upper back are kept in natural and neutral anatomical alignment to prevent strain and pain that a user can otherwise suffer from when he or she is laying on his or her back.

In certain embodiments, the second inflatable airbags that can be positioned in the second chambers define side portions of the neck support device on which the head, face and neck of a user rest on when he or she is laying on a side of the neck support body. The second inflatable airbags are greater under an uninflated state in length and/or volume than that of the first inflatable airbag under an uninflated state, so as to provide a large surface area that can cover body portions including the majority of the surface area of the side profile of the head and face resting thereon. Each of the second chambers is larger than the first chamber. At least one of the second inflatable airbags has a flat top end that is to face an upper inner surface of a corresponding second chamber and a flat bottom base that is opposite to the flat top end and is to face a bottom opening of the second chamber. The flat bottom base is non-flexible/non-expandable and can minimize the expansion downwards when the second inflatable airbag is inflated by a user. The flat top end is expandable, and when inflated, expands upwards towards the side of the head, face and neck of the user. Each of the second inflatable airbags located in the second chambers and the side portions of the neck support device can support the head and neck of a user laying on a corresponding side portion, for example, a right side portion when he or she rests on the right side of the neck support device, or a left side portion when he or she rests on the left side of the neck support device. Further, the airbags are sized to provide a wide range of support and lift that is suitable for both adults and children, as well as people with different head and neck sizes, with different shoulder width, and different degree of neck stiffness.

In certain embodiments, the top and lateral sides of at least one of the airbags are made with a soft and flexible thermoplastic material to allow easy expansion with low pressure and minimal air leakage over time, and to avoid producing excessive noise made by user movement on the neck support device, which can otherwise disrupt the user's sleep.

In certain embodiments, the inflation portion that inflates the second inflatable airbags positioned in the second chambers is independent from the inflation portion that inflates the first inflatable airbag positioned in the first chamber, or different valves are included in the neck support device, so as to create separate inflation system between the second inflatable airbags and the first inflatable airbag. The separate inflation portions or air valves that create separate inflation system serve to differ the amount of inflation/expansion needed for the airbags, since an amount of inflation/expansion needed for the first inflatable airbag can be, and frequently is, different from that for the second inflatable airbags. When a user inflates a second inflatable airbag so as to arrive at a desired level of height and softness/firmness of a side portion of the neck support device, the second inflatable airbag rise upwards towards the head, face and neck of the user laying on his or her right or left side of the body on the side portion.

In certain embodiments, the inflation portion(s) can be one or more manual pump(s), such as hand press inflation pump(s), or one or more powered pump(s), such as electric inflation pump(s), and the airbags can be inflated by the inflation portion(s) through different pipe-body arrangements, such as the second inflatable airbags being interconnected with pipe bodies so as to be inflated or deflated together at once, while the first inflatable airbag being independent from the pipe-body connection relationship of the second inflatable airbags so that inflation and deflation can be adjusted for the first inflatable airbag independently form the second inflatable airbags.

In certain embodiments, the inflation device includes only one manual pump or powered pump and multiple air valves are assembled to the pipe bodies, so as to control air flow in and out of the different airbags.

In certain embodiments, the airbags are embedded deep inside the first and second chambers inside the neck support device, and each is covered on at least the lateral and top sides by the pillow body, which can be molded foam, to ensure maximum comfort effectiveness. On the contrary, if the neck support device were to be created to include only inflatable airbags with no foam or other materials covering the airbags, the neck support device will not be comfortable for a user and will not conform to the head and neck of the user effectively. Rather, the design of the neck support device according to the present disclosure takes into consideration and addresses the conformability of the airbags, the plasticity of the material(s) of which the neck support device is made, and the orthopedic shape thereof, so as to enable a user to make adjustments easily to the neck support device in order to customize its height, softness/firmness, size and/or shape he or she finds most comfortable while laying on his or her back or sides.

In certain embodiments, an area of the neck support device where the back of the head of a user laying on his or her back and resting on the neck support device is softer than an area that corresponds to the neck of the user, so that the head can sink into the pillow comfortably, while the neck-corresponding area is firmer to provide better support for the neck. For example, the center support body is firmer than a center portion of the bearing portion that corresponds to the back of the head when the user lays his or her neck on the center support body.

In certain embodiments, the area of the neck support device where the back of the head of a user laying on his or her back and resting on the neck support device is softer than the side portions, and the side portions that are firmer can offer more support when a user is laying on his or her sides. For example, the side portions of the pillow body that include corresponding portions of the supporting portion and the bearing portion and correspond to the second chambers are firmer than the rest of the bearing portion.

In certain embodiments, to achieve the firmness difference, multiple holes can be punctured in the pillow body. A central region of the neck support device where the back of the head rests on has larger holes thereon to make the material(s) of the pillow body softer and to deform to a greater extent under the weight of the head. Other areas of the neck support device where more support is desired can have smaller holes to reduce the softness or possibility of deformation of the pillow body material(s) under the weight of the head in such regions. Such holes punctured in the material(s) of the pillow body also make the pillow more breathable for a user, cooler to lay thereon, and lighter to be moved about. That is, the different sized holes in various regions of the neck support device further increases the self-conforming characteristics of the neck support device to provide more comfort to a user. In certain embodiments, the pillow body can be fitted with a pillowcase that is a removable cloth or fabric fastened with physical therapy portions such as electrode units, or a regular pillowcase. The physical therapy portions are positioned to match at least one of the positions at the back of the neck and upper shoulders of the user when he or she is laying on the neck support device in a supine position. For a user with neck and shoulder pain and wishing to have electrotherapy, the removable cloth or fabric fastened with physical therapy portions in such positions according to the present disclosure enables the user to have targeted electrotherapy easily for symptoms such as headaches, and/or neck and shoulder pain, without having to know where to place the physical therapy portions at the back of his or her neck and upper shoulders for effective treatment. The physical therapy portions on the pillowcase are positioned to stimulate the nerves and muscles of the upper neck and of the lower neck and upper shoulder junction. These physical therapy portions can be applied with or made of electrical conductive materials and/or sticky gel, and can be connected and be used with an electrotherapy device used for relieving muscle tension and pain.

In certain embodiments, the first inflatable airbag can expand to a maximum expansion state with a maximum height of the first inflatable airbag in a direction of the displacement axis being 5.5 cm to 9.5 cm.

In certain embodiments, at least one of the second inflatable airbags can expand to a maximum expansion state with a maximum height of the second inflatable airbag in a direction of the displacement axis being 6.5 cm to 10.5 cm.

In certain embodiments, at least one of the physical therapy portions is fastened to a position on the pillowcase that corresponds to a position of the center support body and can abut against the neck of the user and be electrically connected to a physical therapy device to receive electric power transmitted from the physical therapy device.

In certain embodiments, at least one of the physical therapy portions is arranged with an electrotherapy unit that can receive the electric power transmitted from the physical therapy device and output electrical stimulation to the neck of the user.

In certain embodiments, at least one of the physical therapy portions is arranged with a heat therapy unit that can receive the electric power transmitted from the physical therapy device and generate heat to heat the neck of the user.

In certain embodiments, at least one of the physical therapy portions is arranged with a combined electrotherapy-heat therapy unit that can receive the electric power transmitted from the physical therapy device, output electrical stimulation to the neck of the user and generate heat to heat the neck.

In certain embodiments, the first inflatable airbag and the second inflatable airbags are arranged within the pillow body, the inflation device is external to the pillow body and the pillowcase, and the pipe bodies can extend into the pillow body through the pillowcase.

Another aspect of the present disclosure is directed to a method for manufacturing an adjustable inflatable neck support device. The method includes forming a pillow body having a supporting top surface that can support a neck of a user, a bearing top surface that can bear an occiput of a user when the user is in a supine position, and a bottom surface formed with a first chamber and two second chambers located at opposite sides of the first chamber, wherein a height of the bearing top surface is smaller than a height of the supporting top surface; forming a bottom plate having edges matching bottom inner edges of the pillow body; positioning the first inflatable airbag in the first chamber, positioning the two second inflatable airbags in the second chambers, respectively, and assembling the pipe bodies to the first inflatable airbag and the second inflatable airbags; and assembling the pillow body with the bottom plate by adhering the bottom inner edges of the pillow body to the matching edges of the bottom plate to form the adjustable inflatable neck support device with the first inflatable airbag and second inflatable airbags enclosed within the adjustable inflatable neck support device.

This and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a schematic view of the neck support device according to the present disclosure.

FIG. 2 is a top view of the neck support device showing the relative positions of the first and second inflatable airbags according to the present disclosure.

FIG. 3 is a side view showing a user resting in a supine position on the neck support device according to the present disclosure.

FIG. 4 is a top view of a user resting in a supine position on the neck support device according to the present disclosure.

FIG. 5 is a top view showing a user resting on the side of his or her head on the neck support device according to the present disclosure.

FIG. 6 is a side view of a user resting on the side of his or her head on the neck support device according to the present disclosure.

FIG. 7 is a top exploded view of the neck support device according to the present disclosure.

FIG. 8 is a bottom exploded view of the neck support device according to the present disclosure.

FIG. 9 is a schematic diagram showing the inflation change of a first inflatable airbag of the neck support device according to the present disclosure.

FIG. 10 is a schematic diagram showing the inflation change of second inflatable airbags of the neck support device according to the present disclosure.

FIG. 11 is a top view of the neck support device according to certain other embodiments of the present disclosure.

FIG. 12 is a perspective view of the neck support device according to certain other embodiments of the present disclosure.

FIG. 13 is another perspective view of the neck support device according to certain other embodiments of the present disclosure.

FIG. 14 is an assembled view showing a physical therapy device being harnessed in a holder by a secure releasable locking mechanism according to certain embodiments of the present disclosure.

FIG. 15 is an exploded view of the assembly of the physical therapy device and the holder according to certain embodiments of the present disclosure.

FIGS. 16 and 17 are exploded views showing the detailed components of the physical therapy device, the secure releasable locking mechanism and the holder according to certain embodiments of the present disclosure.

FIG. 18 is an assembled view showing the physical therapy device being harnessed in the holder according to certain other embodiments of the present disclosure.

FIGS. 19 and 20 are exploded views of the assembly of the physical therapy device and the holder according to certain other embodiments of the present disclosure.

FIG. 21 is an assembled view showing the physical therapy device being harnessed in the holder according to yet certain other embodiments of the present disclosure.

FIG. 22 is an exploded view of the assembly of the physical therapy device and the holder according to yet certain other embodiments of the present disclosure.

FIG. 23 is an exploded view showing the physical therapy device and the detailed components of the holder according to yet certain other embodiments of the present disclosure.

FIG. 24A is a schematic view of an electrotherapy unit arranged at a physical therapy portion according to the present disclosure.

FIG. 24B is a schematic view of a heat therapy unit arranged at a physical therapy portion according to the present disclosure.

FIG. 24C is a schematic view of a combined electrotherapy-heat therapy unit arranged at a physical therapy portion according to the present disclosure.

FIG. 25 is a flowchart showing the steps of a method for manufacturing the neck support device.

### DETAILED DESCRIPTION

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, parts or the like, which are for distinguishing one component/part from another one only, and are not intended to, nor should be construed to impose any substantive limitations on the components, parts or the like, or be relevant to the sequence in which the components/parts are to be assembled or disposed in practical application.

As used herein, the term “substantially” or “approximately” refers to, for example, a value, or an average of values, in an acceptable deviation range of a particular value recognized or decided by a person of ordinary skill in the art, taking into account any specific quantity of errors related to the measurement of the value that may resulted from limitations of a measurement system or device. For example, “substantially” may indicate that the value is within, for example, ±5%, ±3%, ±1%, ±0.5% or ±0.1%, or one or more standard deviations, of the particular value.

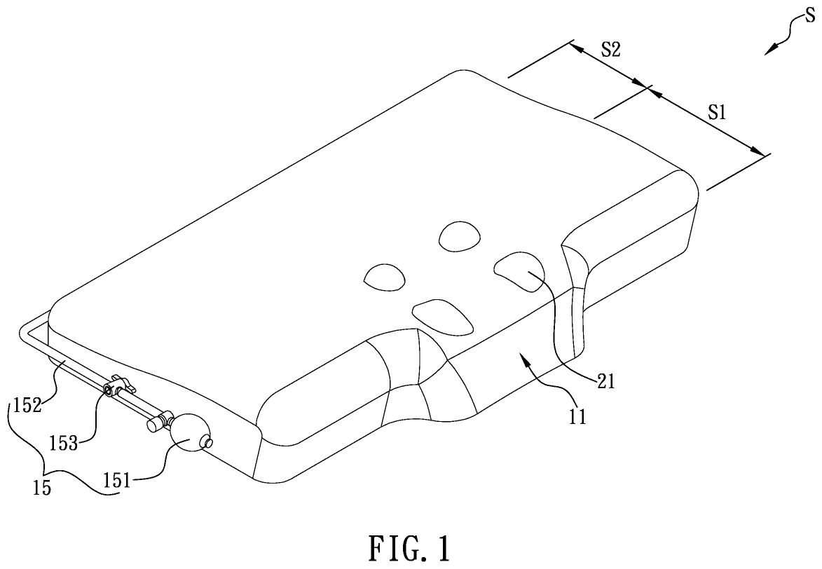

One aspect of the present disclosure is directed to an adjustable inflatable neck support device. To facilitate understanding only, unless the context clearly dictates otherwise, the lower right side of FIG. 1 is designated as the front sides of the components shown in FIG. 1, the upper left side of FIG. 1 is designated as the rear sides of the components, the upper right side of FIG. 1 is designated as the right sides of the components, the lower left side of FIG. 1 is designated as the left sides of the components, the top side of FIG. 1 is designated as the top sides of the components, and the bottom side of FIG. 1 is designated as the bottom sides of the components. However, the present disclosure is not limited thereto. In certain embodiments, an adjustable inflatable neck support device S can be divided from the front to the rear at least into a supporting portion S1 and a bearing portion S2. The shape of the top surfaces of the supporting portion S1 and the bearing portion S2 is ergonomically designed to fit the natural curvature of the cervical spine, so that the height of the top surface of the bearing portion S2 is lower than the height of the top surface of the supporting portion S1.

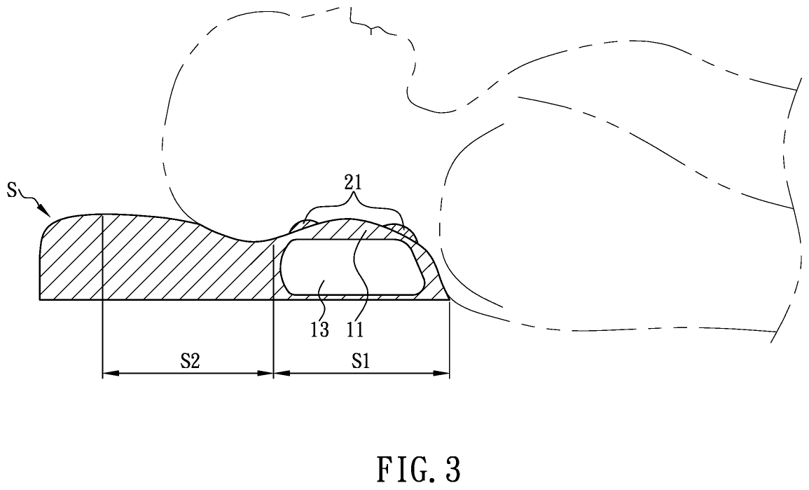

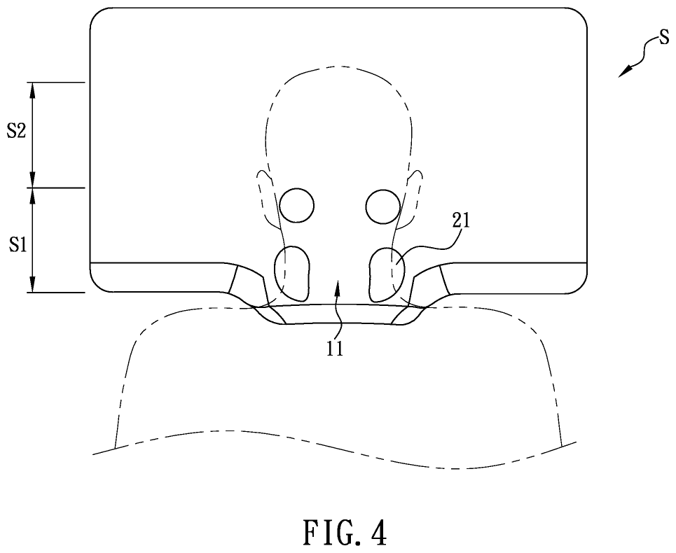

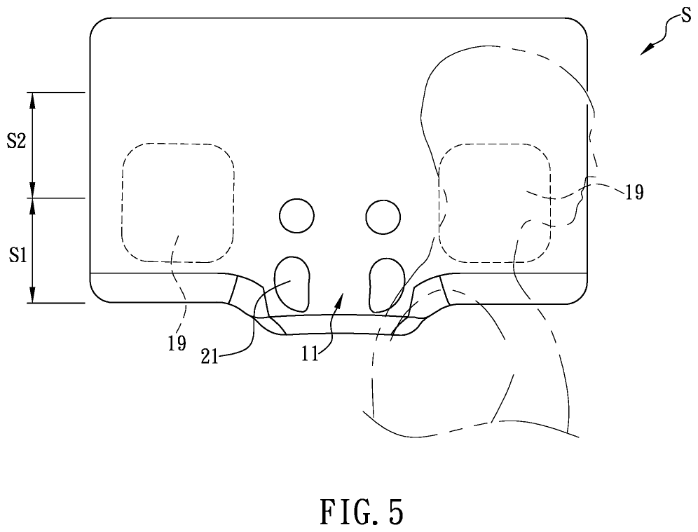

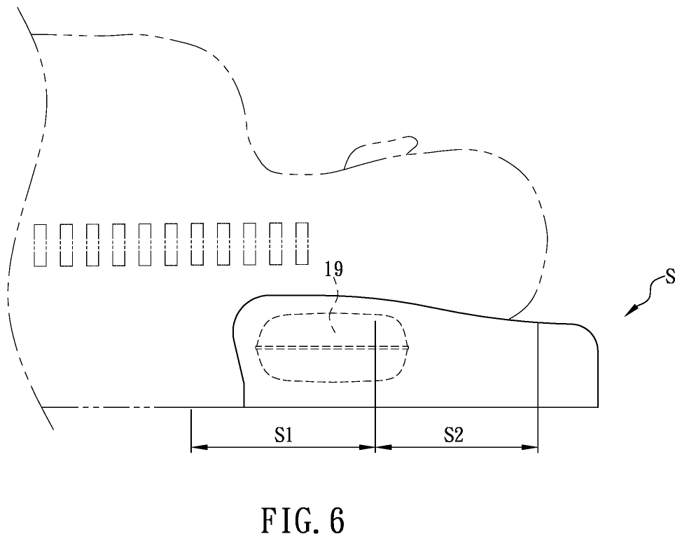

Referring to FIGS. 1, 3 and 4, when a user rests on the neck support device S in a supine position, the bearing portion S2 corresponds to and bears the occiput of the user (as shown in FIG. 3), while the supporting portion S1 corresponds to and supports the neck of the user. Referring to FIGS. 5 and 6, when a user rests sideways on the neck support device S, the bearing portion S2 corresponds to and bears the lateral side of the head of the user (as shown in FIG. 6), while the supporting portion S1 corresponds to and supports the lateral side of the neck of the user. The contour of the supporting portion S1 matches the natural cervical lordosis of the human cervical spine to provide corresponding support force, so as to help the user to restore the alignment and posture of his or her head, cervical spine and upper thoracic spine to the natural states. In addition, because the neck support device S has elasticity, when a user rests on the neck support device S, the neck support device S deforms so that the top surface fits the contour of the head and neck of the user, and achieves the bearing and supporting effects.

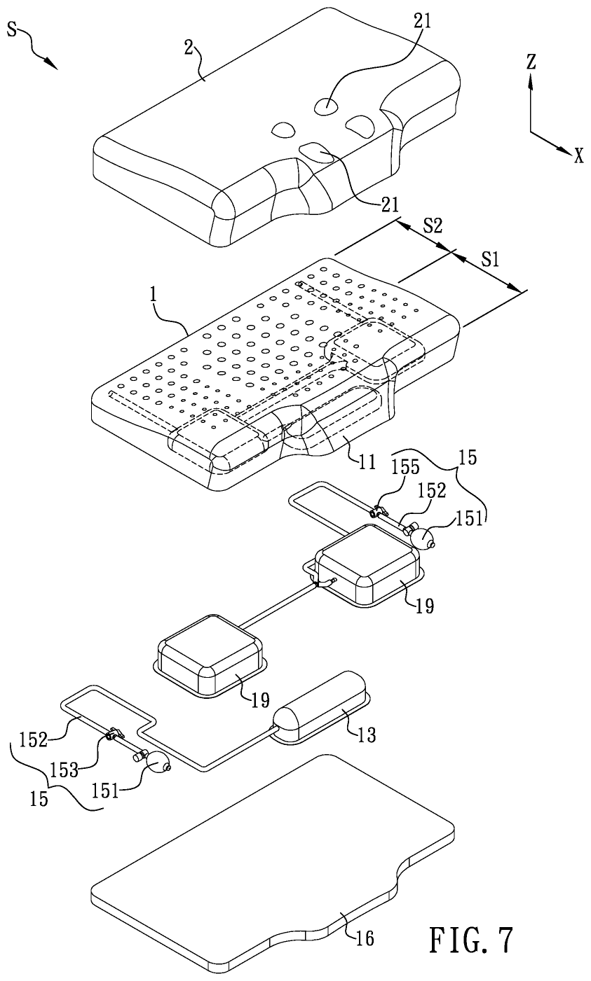

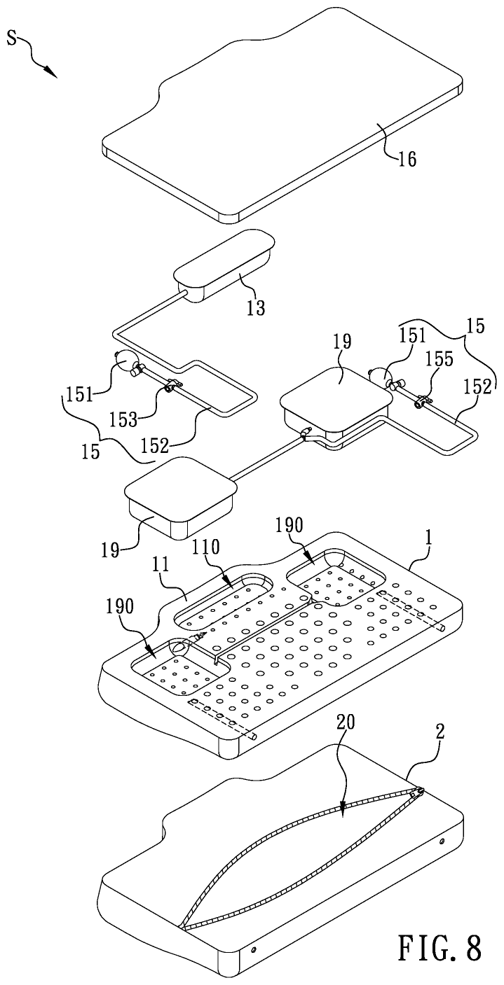

Referring to FIGS. 7 and 8, the neck support device S includes a pillow body 1 and a pillowcase 2. The pillowcase 2 is provided with an accommodating space 20 and is configured to cover the pillow body 1, so that the pillow body 1 can be positioned in the accommodating space 20. The pillowcase 2 can be a removable cloth or fabric fastened with physical therapy portions 21, such as electrode units, and having a profile matching the contour of the pillow body 1. However, the present disclosure is not limited thereto. In certain embodiments, the pillowcase can be a regular pillowcase. As the contour of the pillowcase 2 matches that of the pillow body 1, when the pillow body 1 is placed in the pillowcase 2, the structure of the pillow body 1 can still be clearly indicated through the contour of the pillowcase. Accordingly, in FIG. 1, for ease of description only, the positions of the components of the pillow body 1 to be described infra are also indicated on the corresponding positions on pillowcase 2.

Referring again to FIGS. 7 and 8, in certain embodiments, the pillow body 1 can be made of foamed material and/or other elastomer, and/or filling/packing material, and have the supporting portion S1 and the bearing portion S2. The center portion of the supporting portion S1 is provided with a center support body 11. The top surface of the center support body 11 can support the neck of the user (as shown in FIG. 3). In addition, in order to better achieve the effect of neck supporting, the front side of the center support body 11 extends forward, so as to completely or nearly completely support the user's neck. Accordingly, when the center support body 11 abuts and supports the user's neck, the head of the user will naturally tilt backward, which helps to fix more stably the head of the user to the bearing portion S2. In certain embodiments, the center support body 11 that corresponds to the neck of a user is firmer than a center portion of the bearing portion S2 that corresponds to the back of the head of the user when he or she lays his or her neck on the center support body 11. The firmness difference between the center support body 11 and the center portion of the bearing portion S2 allows the head to sink into the pillow body 1 comfortably while sufficient support can be provided to the neck by the center support body 11. In certain embodiments, the firmness difference is produced by density, total opening area, diameter and/or location differences of the holes punctured on the pillow body 1. For example, a foam material of the center portion of the bearing portion S2 can have holes in greater density, larger holes and/or holes with greater diameters than the holes punctuated on the center support body 11, so that the center portion of the bearing portion S2 is softer than the center support body 11, and the center support body 11 is of greater supportability. In certain embodiments, the center support body 11 is firmer than the entire bearing portion S2.

As shown in FIGS. 7 and 8, the bottom surface of the center support body 11 can be concavely provided with a first chamber 110, and a first inflatable airbag 13 can be accommodated in the first chamber 110. At least the upper inner surface of the first chamber 110 is expandable for a first upward maximum expansion degree to allow the first inflatable airbag 13 adequate amount of space needed for various levels of expansion as desired by a user when he or she inflates the first inflatable airbag 13 to customize the level of firmness/softness and height of the corresponding top surface of the neck support device S for his or her unique requirements. The first upward maximum expansion degree can be 5-9 cm, and in certain embodiments, 6.5-7.5 cm. The bottom opening of the first chamber 110 may be covered by a bottom plate, for example, a bottom plate 16 shown in FIG. 8, which can be made of plastic, metal or other material that is non-expandable, minimal-expandable, or less expandable than foam materials, so that the majority of the expansion of the first inflatable airbag 13 when inflated is at the upper side of the first inflatable airbag 13 that faces the top surface of, and is in a direction towards the neck and head of a user resting on, the neck support device S, restricting expansion downwards towards the bottom of the neck support device S, and therefore restricting the excessive outward expanding forces against the bottom structure of the neck support device S, which can cause damage to the neck support device S, and directing, collectively with the expandable upper inner surface of the first chamber 110, the expansion upwards. In certain embodiments, the first chamber 110 can be formed by hollowing out an accommodating space from a molded foam that, with the first upward maximum expansion degree, allows room for the first inflatable airbag 13 to expand in various levels. In addition, it is noted that as long as the first chamber 110 can accommodate the first inflatable airbag 13, and allow the first inflatable airbag 13 to expand or contract to displace the top surface of the center support body 11, the first chamber 110 is defined as being located within the bottom surface of the center support body 11. The first inflatable airbag 13 can be inflated to expand, or deflated to contract, along a displacement axis (for example, the Z axis shown in FIG. 7). In certain embodiments, an uninflated first inflatable airbag 13 has a volume of 9-21 cm³, in certain embodiments, 14-17 cm³, the top and bottom sides of the uninflated first inflatable airbag 13 respectively abut against the top inner surface and flush with the bottom opening, that is, also abutting against a bottom plate 16 if the bottom plate 16 covers the bottom opening, of the first chamber 110 along the displacement axis, and the lateral sides of the uninflated first inflatable airbag 13 respectively abut against the four lateral inner surfaces of the first chamber 110 along a chamber longitudinal axis of the first chamber 110 that is perpendicular or substantially perpendicular to the displacement axis and parallel or substantially parallel to a front-rear direction of the neck support device S, and along a chamber transverse axis of the first chamber 110 that is perpendicular or substantially perpendicular to the chamber longitudinal axis and parallel or substantially parallel to a right-left direction of the neck support device S. By abutting against the inner surfaces of the first chamber 110 under an uninflated state, the expansion of the first inflatable airbag 13 more efficiently drives the expansion of the first chamber 110 by lowering delayed expansion correspondence therebetween, which can otherwise occur when gaps exist between the first chamber 110 and an uninflated first inflatable airbag 13. However, the present disclosure is not limited thereto, and in certain embodiments, at least one of the top and bottom sides of the uninflated first inflatable airbag 13 does not abut against the top inner surface or flush with the bottom opening of the first chamber 110 along the displacement axis, and/or at least one of the lateral sides of the uninflated first inflatable airbag 13 does not abut against any of the four lateral inner surfaces of the first chamber 110 along the chamber longitudinal axis or the chamber transverse axis of the first chamber 110.

When the first inflatable airbag 13 expands or contracts along the displacement axis, the expansion or contraction displaces the top surface of the center support body 11 along the displacement axis, for example, upward or downward. In certain embodiments, the first inflatable airbag 13 is semi-tubular in shape and has a rounded first end that is to face the upper inner surface of the first chamber 110, and an opposite second end that is a flat base that is to face the bottom opening of the first chamber 110. In certain embodiments, the flat base is non-flexible or minimal-expandable, and when the first inflatable airbag 13 is inflated, an expansion degree along the displacement axis of the first end is greater than that of the second end. When the first inflatable airbag 13 is inflated, the first inflatable airbag 13 can expand upwards towards the posterior side of the cervical spine of the user when he or she is laying in a supine position on the center support body 11 of the neck support device S, and drive the displaced corresponding top surface of the center support body 11 upwards to match the anatomical shape of the cervical spine, for example, conform against a slightly lordotic curvature of the cervical spine of a user. In certain embodiments, when the first inflatable airbag 13 is inflated to expand to a first maximum expansion state, the maximum height of the first inflatable airbag 13 in the direction of the displacement axis is 5.5 cm to 9.5 cm, or in certain embodiments, 7 cm to 8 cm. However, the present disclosure is not limited thereto. Accordingly, when a user inflates the first inflatable airbag 13 to a level of height and softness/firmness that provides him or her with the desired customization, with the aid of the inflated first inflatable airbag 13 and the material(s) which the neck support device S is made of and holds the inflated first inflatable airbag 13, the top surface of the neck support device S that corresponds to the inflated first inflatable airbag 13 can conform comfortably against the curvature of the cervical spine of a user when he or she is laying in a supine position. Further, with the combined effects provided by the adjustable inflatable airbag 13 being within the material of the neck support device S, for example, orthopedic shaped foam, customized support for the head and neck of a user can be provided to keep the head, neck and upper back in natural and neutral anatomical alignment so as to prevent strain and pain when the user is laying on his or back. Referring again to FIGS. 7 and 8, in certain embodiments, the neck support device S further includes at least two inflatable second inflatable airbags 19 in addition to the first inflatable airbag 13. Each of the inflatable second inflatable airbags 19 in an uninflated state is larger than the inflatable first inflatable airbag 13 in an uninflated state, for example, a maximum cross-sectional area perpendicular or substantially perpendicular to the displacement axis of an inflatable second inflatable airbags 19 in an uninflated state is larger than that of the inflatable first inflatable airbag 13 in an uninflated state by 120-170 cm², or in certain embodiments, by 135-155 cm², or by 5-15 cm in length along a corresponding chamber longitudinal axis, or in certain embodiments, 8.5-11.5 cm in length along a corresponding chamber longitudinal axis, and has a maximum expanded volume greater than that of the inflatable first inflatable airbag 13, for example, by 720-1800 cm³, so as to displace a larger area on the top surface of the neck support device S than an area on the top surface of the neck support device S displaceable by the first inflatable airbag 13 to cover a majority, for example, more than 50%, of the surface area of the side profile of the head, face and neck of a user when he or she sleeps on his or her side on a side of the neck support device S. The bottom surface of the pillow body 1 can be concavely provided with two second chambers 190 at two opposite sides of the first chamber 110 and adjacent to the opposite ends of the pillow body 1, respectively, for example, one adjacent to a right side of the pillow body 1, and the other one adjacent to the left side of the pillow body 1, that is, one nearer to the right side of the pillow body 1 than to the left side thereof, and the other one nearer to the left side of the pillow body 1 than to the right side thereof. Each second chamber 190 has a chamber longitudinal axis that is perpendicular or substantially perpendicular to the displacement axis and parallel or substantially parallel to a front-rear direction of the neck support device S and a chamber transverse axis that is perpendicular or substantially perpendicular to the chamber longitudinal axis and parallel or substantially parallel to a right-left direction of the neck support device S. In certain embodiments, at least the length along the chamber longitudinal axis of an inflatable second inflatable airbags 19 in an uninflated state is greater than that of the inflatable first inflatable airbag 13 in an uninflated state, for example, by 6-15 cm, or in certain embodiments, by 8.5-11.5 cm. Each of the second chambers 190 has an upper inner surface whose area is larger than that of the upper inner surface of the first camber 110, and a length along the chamber longitudinal axis that is greater than that of the of the first camber 110, so as to allow for a larger displacement area on the top surface of the neck support device S than an area on the top surface of the neck support device S displaceable by the first inflatable airbag 13 in order to cover a majority, for example, more than 50%, of the surface area of the side profile of the head, face and neck of a user when he or she sleeps on his or her side on a side of the neck support device S. Each of the second chambers 190 can be located entirely within the supporting portion S1, so that areas of the top surface of the neck support device S corresponding in position to the second chambers 190 can be displaced by the expansion or contraction of the second inflatable airbags 19 accommodated in the second chambers 190 to adjust the height of such portions to suit the height of the neck of a user resting on his or her side and provide proper support to his or her cervical spine. However, the present disclosure is not limited thereto. In certain embodiments, at least one of the second chambers 190 is located both in the supporting portion S1 and the bearing portion S2, with a first portion of such a second chamber 190 located in the supporting portion S1 being the same as or greater than a second portion of such a second chamber 190 located in the bearing portion S2, so that the height of an area of the top surface of the neck support device S corresponding in position to such a second chambers 190 can be adjusted to suit the heights both of the neck and the head of a user resting on his or her side, and provide proper support to and maintain the neutral anatomical spinal alignment of his or her head, cervical spine and upper thoracic spine without the head being tilted to an excessive extent. In certain embodiments, a first portion of a second inflatable airbag 19 that is located in the supporting portion S1 has a first maximum inflated height of 6.5-10.5 cm, or in certain embodiments, 8.0-9.0 cm. A second portion of the second inflatable airbag 19 that is located in the bearing portion S2 has a second maximum inflated height of 5.5-9.5 cm, or in certain embodiments, 7.0-8.0 cm. In certain embodiments, the first maximum inflated height is greater than the second maximum inflated height, and a ratio of the first maximum inflated height to second maximum inflated height can be between 1.11:1 and 1.90:1.

In certain embodiments, the side portions of the pillow body 1 where the second chambers 190 are located, consisting of corresponding portions of the supporting portion S1 and the bearing portion S2, are firmer than the rest of the bearing portion S2, and have a firmness equal to or higher than that of the center support body 11, so as to offer more support when a user is laying on his or her sides on a side of the neck support device S. In certain embodiments, a portion of the bearing portion S2 in a side portion where a second chamber 19 is located is softer than a portion of the supporting portion S1 in the side portion, so as to allow the head to sink into the pillow body 1 comfortably while sufficient support can be provided to the neck from the portion of the supporting portion S1 in such a side portion. Such firmness difference between a side portion and the rest of the bearing portion S2, and between a portion of the bearing portion S2 in a side portion and a portion of the supporting portion S1 in the side portion can be produced by density, total opening area, diameter and/or location differences of the holes punctured on the pillow body 1. In certain embodiments, the material of the bearing portion S2 excluding the side portions can have holes in greater density, larger holes and/or holes with greater diameters than the holes punctuated on the side portions, so that the bearing portion S2 excluding the side portions is softer than the side portions, and the side portions are of greater supportability. In certain embodiments, the material of a portion of the bearing portion S2 in a side portion can have holes in greater density, larger holes and/or holes with greater diameters than the holes punctuated on a portion of the supporting portion S1 in the side portion, so that the portion of the bearing portion S2 in the side portion is softer than the portion of the supporting portion S1 in the side portion, and the portion of the supporting portion S1 in the side portion are of greater supportability. In certain embodiments, the top surface of a portion of the supporting portion S1 that is located in a side portion has a first maximum displacement of 2.1-5.7 cm, and in certain embodiments, 3.5-4.3 cm. The top surface of a portion of the bearing portion S2 that is located in the side portion has a second maximum displacement of 1.4-5.0 cm, and in certain embodiments, 2.9-3.5 cm. In certain embodiments, the first maximum displacement is greater than the second maximum displacement, and a ratio of the first maximum displacement to the second maximum displacement is between 1.14:1 and 4.07:1.

Referring again to FIGS. 7 and 8, the second inflatable airbags 19 can be accommodated in the second chambers 190, respectively, and be inflated to expand, or deflated to contract, along the displacement axis (for example, the Z axis shown in FIG. 7). At least the upper inner surface of each of the second chamber 190 is expandable for a second upward maximum expansion degree to allow the respective second inflatable airbags 19 adequate amount of space needed for various levels of expansion as desired by a user when he or she inflates the second inflatable airbags 19 to customize levels of firmness/softness and height of the corresponding top surface of the neck support device S for his or her unique requirements. The second upward maximum expansion degree can be 6-10 cm, or in certain embodiments, 7.5-8.5 cm. In certain embodiments, only the top and lateral sides of at least one of the first and second inflatable airbags 13, 19 are expandable, and the bottom side thereof is non-expandable or minimal-expandable. For example, only the top and lateral sides of the first and second inflatable airbags 13, 19 are made of a soft and flexible thermoplastic material to allow quick expansion towards the upward and lateral directions even in response to low-pressure inflation, which lowers the risk of air leakage over time as can occur when an airbag has a relatively higher inner pressure, and lowers the possibility of noise and, if any, the volume thereof, that may be generated by user movement on the neck support device S, which can otherwise disrupt user sleep. The bottom opening of each second chamber 190 may be covered by a bottom plate, for example, the bottom plate 16 shown in FIG. 8, which can be made of plastic, metal or other material that is non-expandable, minimal-expandable, or less expandable than foam materials, so that the majority of the expansion of the second inflatable airbags 19 when inflated is at the upper side of the second inflatable airbags 19 that faces the top surface of, and is in a direction towards the neck and head of a user resting on, the neck support device S, restricting expansion downwards towards the bottom of the neck support device S, and therefore restricting the excessive outward expanding forces against the bottom structure of the neck support device S, and directing, collectively with the expandable upper inner surface of the second chamber 190, the expansion upwards. In certain embodiments, the second chambers 190 can be formed by hollowing out accommodating spaces from a molded foam that, with the second upward maximum expansion degree, allows room for the second inflatable airbags 19 to expand in various levels. In certain embodiments, an uninflated second inflatable airbag 19 has a volume greater than that of an uninflated first inflatable airbag 13, and is of 25-36 cm³, or in certain embodiments, 28-33 cm³, the top and bottom sides of the uninflated inflatable airbag 19 respectively abut against the top inner surface and flush with the bottom opening, that is, also abutting against a bottom plate 16 if the bottom plate 16 covers the bottom opening, of a corresponding second chamber 190 along the displacement axis, and the lateral sides of the uninflated inflatable airbag 19 respectively abut against the four lateral inner surfaces of the corresponding second chamber 190 along the chamber longitudinal axis and the chamber transverse axis of the second chamber 190. By abutting against the inner surfaces of the second chamber 190 under an uninflated state, the expansion of the inflatable airbag 19 more efficiently drives the expansion of the second chamber 190 as delayed expansion correspondence therebetween is lowered, which can otherwise occur when gaps exist between the second chamber 190 and an uninflated second inflatable airbag 19. However, the present disclosure is not limited thereto, and in certain embodiments, at least one of the top and bottom sides of the uninflated second inflatable airbag 19 does not abut against the top inner surface or flush with the bottom opening of the second chamber 190 along the displacement axis, and/or at least one of the lateral sides of the uninflated second inflatable airbag 19 does not abut against any of the four lateral inner surfaces of the second chamber 190 along the chamber longitudinal axis and the chamber transverse axis of the second chamber 190.

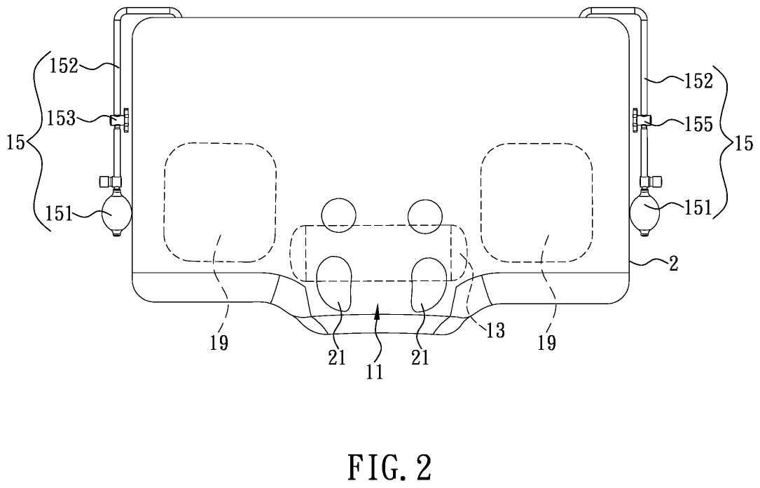

When a second inflatable airbag 19 expands or contracts along the displacement axis, the expansion or contraction displaces the top surface of the pillow body 1, and therefore also the top surface of the pillowcase 2, along the displacement axis, for example, upward or downward, which can help to further block the head and shoulder of a user from sliding sideways and away from the center support body 11 due to the movement of the first inflatable airbag 13 during the usage of the neck support device S, so as to better anchor the head and neck of a user at the position on the neck support device S that corresponds to the first inflatable airbag 13, which position is shown in FIG. 2 for reference, and with such anchoring also maintain proper positioning of the shoulders of the user relative to the neck support device S, so that excellent electrotherapy, heat therapy and/or traction effects can be ensured. In certain embodiments, at least one of the second inflatable airbag 19 has a flexible flat top end that is to face the upper inner surface of a corresponding second chamber 190, and an non-flexible or minimal-expandable flat bottom base that is opposite to the flat top end and to face the bottom opening of the second chamber 190, and when the second inflatable airbag 19 is inflated, an expansion degree along the displacement axis of the flexible flat top end is greater than the non-flexible flat bottom base. In certain embodiments, when a second inflatable airbag 19 is inflated to expand to a second maximum expansion state, the maximum height of the second inflatable airbag 19 in the direction of the displacement axis is 6.5 cm to 10.5 cm, or in certain embodiments, 8 cm to 9 cm. However, the present disclosure is not limited thereto. Further, the maximum height of one of the second inflatable airbags 19 can be the same or different from that of the other one of the second inflatable airbags 19, so that greater flexibility in height adjustment can be afforded to the different sides of the neck support device S to a user.

Further, the addition and arrangement of the second inflatable airbags 19 to and for the neck support device S can also enable a user who is used to sleep on his or her side, that is, a side sleeper, to adjust the height and firmness of the side areas of the neck support device S on which the side sleeper rests his or her side of the head when laying sideways on the neck support device S. While a user of the neck support device S according to the present disclosure who is accustomed to sleeping on his or her back, that is, a back sleeper, can adjust the height and firmness of the area of the neck support device S that corresponds to the center support body 11 and the first inflatable airbag 13 and to his or her neck when he or she is sleeping on his back, by inflating or deflating, and changing the expansion or contraction degree of, the first inflatable airbag 13, so as to better support his or her cervical spine and restore the lordotic curvature of his or her cervical spine to a natural state, the height and firmness adjustment of the center support body 11 and the first inflatable airbag 13 may not be sufficiently satisfactory in terms of head and cervical spine support and head, cervical spine and upper thoracic spine alignment and maintenance for a side sleeper who is more used to lay sideways on the neck support device S and on an area of the neck support device S that is away from the center support body 11 and the first inflatable airbag 13. Moreover, a side sleeper may even need extra head and neck support and spine alignment restoration and maintenance than a back sleeper does due to a greater vertical distance between the shoulder and the head/neck when he or she is in a side-sleeping posture, for example, sleeping on the side of his or her head, than between the back and the head/neck when a back sleeper is in a back-sleeping posture. Referring again to FIGS. 7 and 8, the neck support device S with the second inflatable airbags 19 provides the further head and cervical spine support and head, cervical spine and upper thoracic spine alignment restoration and maintenance needed by a side sleeper, as the side sleeper may also adjust, in addition to the height and firmness of the area of the neck support device S that corresponds to the center support body 11 and the first inflatable airbag 13, the height and firmness of the side areas of the neck support device S, for example, areas of the top surface of the pillow body 1 and of the top surface of the pillowcase 2 that are adjacent respectively to the right and left sides of the pillow body 1 and the pillowcase 2 and corresponding to, and can be displaced by the expansion or contraction of, the second inflatable airbags 19, by inflating or deflating, and changing the expansion or contraction degree of, the second inflatable airbags 19 when the side sleeper rests the side of his or her head on these side areas of the neck support device S. When a second inflatable airbag 19 is inflated, the second inflatable airbag 19 can expand upwards towards the lateral side of the head, cervical spine and upper thoracic spine of the user when he or she is laying on his or her side and on a side of the neck support device S, and drive the displaced top surface of the neck support device S that corresponds to the second inflatable airbag 19 upwards to align the head, cervical spine and upper thoracic spine of a user in a straight line. When the user inflates the second inflatable airbag(s) 19 to a level of height and softness/firmness that provides him or her with the desired customization, with the combined effects provided by the inflated second inflatable airbag(s) 19 and the material(s) which the neck support device S is made of and holds the inflated second inflatable airbag 19, the top surface of the neck support device S that corresponds to the inflated second inflatable airbag(s) 19 can conform comfortably against the contour of the lateral sides of the head, cervical spine and upper thoracic spine, and align the head, cervical spine and upper thoracic spine of a user who is a side sleeper. That is, a side sleeper can adjust the height and firmness of the area of the neck support device S that supports the side of the head when he or she is sleeping on his or her side, so as to arrive at a height and firmness that he or she deems proper for supporting his or her head and neck and to maintain and restore his or her head, cervical spine and upper thoracic spine in alignment and neutral position. However, the present disclosure is not limited thereto. Further, with the combined effects of the adjustable inflatable airbag 19 within the orthopedic shaped foam, for example, orthopedic shaped memory foam, customized support for the head and neck of a user can be provided to keep his or her head, neck and upper back in their natural neutral anatomical alignment so as to prevent strain and pain when the user is laying on his or her side. Accordingly, with the features of and advantages provided by the first inflatable airbag 13 and the second inflatable airbags 19, the neck support device S is suitable for both back sleepers and side sleepers as an everyday sleeping pillow.

Referring again to FIGS. 7 and 8, an inflation device 15 can be connected respectively to the first inflatable airbag 13 and second inflatable airbags 19 which are independent from each other. When the inflation device 15 is in operation, it can inflate or deflate the first inflatable airbag 13 and the two second inflatable airbags 19, and change the degrees of the expansion or contraction of the first inflatable airbag 13 and the two second inflatable airbags 19. In certain embodiments, the inflation device 15 includes at least one inflation portion 151, a plurality of pipe bodies 152, a first air valve 153, and a second air valve 155. The inflation portion 151 can be a hand press inflation pump. The pipe bodies 152 can pass through the pillowcase 2 and the pillow body 1 to communicate with the first inflatable airbag 13 and the two second inflatable airbags 19, respectively, so that the inflation portion 151 can inflate or deflate the first and/or second inflatable airbags 13, 19 through the pipe bodies 152. The first air valve 153 can be assembled to one of the pipe bodies 152, and the pipe body 152 can communicate with the first airbag 13, while the second air valve 155 can be assembled to another pipe body 152 that is to communicate with the second inflatable airbags 19. However, the present disclosure is not limited thereto. In certain other embodiments the inflation portion 151 may be an electric inflation pump, and in certain embodiments, the inflation device 15 includes a plurality of inflation portions 151. Each of the inflation portions 151 can be a hand press inflation pump or an electric inflation pump. A first one of the inflation portions 151 can be connected to at least one of the pipe bodies 152 and communicate with the first inflatable airbag 13 through the pipe bod(ies) 152 corresponding to the inflation portion 151 that corresponds to and can communicate with the first inflatable airbag 13. The first air valve 153 can be assembled to one of the pipe bod(ies) 152 corresponding to the inflation portion 151 that corresponds to and can communicate with the first inflatable airbag 13. A second one of the inflation portions 151 can be connected to at least another one of the pipe bodies 152 and communicate with the second inflatable airbags 19 through the pipe bod(ies) 152 corresponding to the inflation portion 151 that corresponds to and can communicate with the second inflatable airbags 19. That is, an inflation portion 151 that inflates at least one of the second inflatable airbags 19 can be independent from an inflation portion 151 that inflates the first inflatable airbag 13, which inflation portions 151 forming an inflation control system that allows a user to separately control the respective inflation portions 151, so that a degree of inflation of the first inflatable airbag 13 can be controlled by a user to be different and independent from that of the second inflatable airbags 19 through the inflation portions 151, since the degree of, and timing for, inflation needed for the first inflatable airbag 13 can be, and frequently are, different from that needed for the second inflatable airbags 19. The second air valve 155 can be assembled to one of the pipe bod(ies) 152 corresponding to the inflation portion 151 that corresponds to and can communicate with the second inflatable airbags 19.

As shown in FIGS. 7 and 8, when the first air valve 153 is opened, the inflation portion 151 can inflate or deflate the first airbag 13. When the first air valve 153 is closed, the inflation portion 151 would not be able to inflate or deflate the first inflatable airbag 13. Similarly, when the second air valve 155 is opened, the inflation portion 151 can inflate or deflate the two second inflatable airbags 19 at the same time. When the second air valve 155 is closed, the inflation portion 151 would not be able to inflate or deflate the two second airbags 19. In certain embodiments, when one inflation portion 151, either an electric inflation pump or a hand press inflation pump, is connected to the first inflatable airbag 13 and also to the second inflatable airbags 19 to control the volume changes of the first inflatable airbag 13 and the second inflatable airbags 19, the respective volume changes of the airbags can be controlled through the air valves on the pipe bodies 152 corresponding to the airbags 13, 19.

While FIGS. 7 and 8 show only a single second air valve 155 to simultaneously adjust the degrees of inflation or deflation of the two second inflatable airbags 19, in certain embodiments, two second air valves 155 can be provided to independently adjust the degrees of inflation or deflation of any of the second inflatable airbags 19. In addition, in actual use, a user can open both the first air valve 153 and the second air valve 155, so that the inflation portion 151 can inflate the first inflatable airbag 13 and the second inflatable airbags 19 at the same time. A user can also open the first air valve 153 and close the second air valve 155, so as to improve the inflation efficiency of the first inflatable airbag 13. Similarly, a user can open the second air valve 155 and close the first air valve 153, so as to improve the inflation efficiency of the second inflatable airbags 19. However, the present disclosure is not limited thereto, and in certain other embodiments the inflation portions 151 corresponding to the first inflatable airbag 13 and second inflatable airbags 19 can instead be one electric inflation pump that can control the inflation and/or deflation of the first inflatable airbag 13 and/or second inflatable airbags 19 respectively or simultaneously.

In other words, while in certain embodiments the neck support device S include sonly one inflation portion 151, in which air valve(s) can be used to direct the air flow in and out of the first airbag 13 that can be positioned under the neck of a user in a supine position, and be adjusted to allow the air flow be closed off to the first airbag 13 under the neck and be directed to the second airbags 19 on both sides of the neck support device S, in certain embodiments multiple inflation portions 151 can be employed in the neck support device S, so that one inflation portion 151 can direct the air flow to and out of the first airbag 13 under the neck of a user in a supine position, and another inflation portion 151 can direct the air flow in and out of the second airbags 19, so that the inflation portions 151 work independently from one another. Accordingly, the neck support device S may adopt a single-inflation portion system or a multiple-inflation portion system, and in certain embodiments, in either system the air flow to and out of the first airbag 13 can still be separately from that to and out of the second airbags 19, and inflating or deflating the first airbag 13 under the user's neck can still have no effect on the amount of air in the second airbags 19, and inflating or deflating the second airbag(s) 19 under the user's neck can still have no effect on the amount of air in the first airbag 13. However, the present disclosure is not limited thereto.

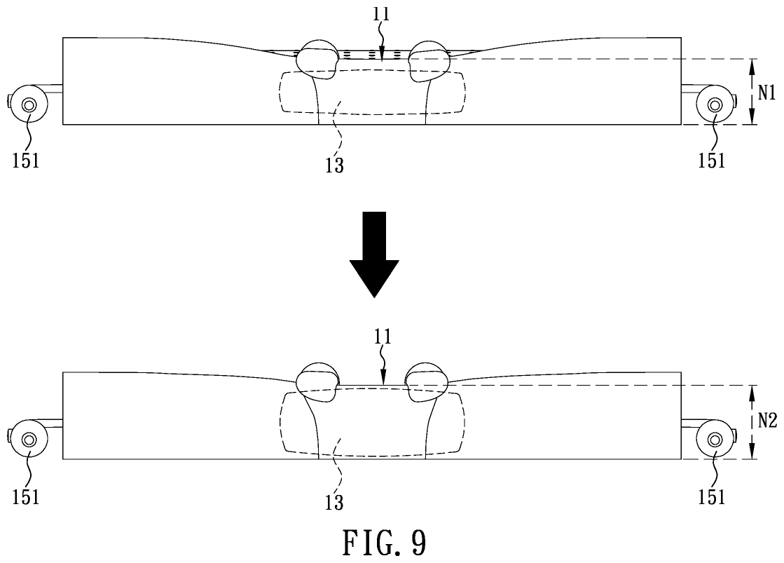

Referring to FIGS. 1, 3 and 9, when the head of a user rests on the neck support device S, the first airbag 13 can be inflated through the inflation portion 151. At this time, the first airbag 13 gradually expands, so that the top surface of the center support body 11 is gradually moved upward, thereby pushing the user's neck upward, and the user's cervical spine can return to its natural lordotic curvature, and present an arch shape. The height of the top surface, or more precisely a point of the top surface that corresponds to the highest point of the first inflatable airbag 13 along the Z axis shown in FIG. 9, of the center support body 11 relative to the bottom surface of the center support body 11 when the top surface is not displaced by the expansion of the first airbag 13 can be defined as a first height N1. The height of the top surface, or more precisely a point of the top surface that corresponds to the highest point of the first inflatable airbag 13 along the Z axis shown in FIG. 9, of the center support body 11 relative to the bottom surface of the center support body 11 when the top surface is displaced by the first airbag 13 expanding to the first maximum expansion state can be defined as a second height N2. In certain embodiments, the ratio of the first height N1 to the second height N2 can range between 1:1.10 and 1:1.70, and in certain embodiments, between 1:1.26 and 1:1.46. Accordingly, by the design of the first inflatable airbag 13, the height and the firmness of an area of the neck support device S that abuts against and corresponds to the neck of a user when the user is lying on the neck support device S, or more particularly, when the user is lying on the center support body 11 and on his or her back, can be adjusted to keep the neck of the user at a proper position and allow the head, cervical spine and upper thoracic spine of the user to be aligned properly.

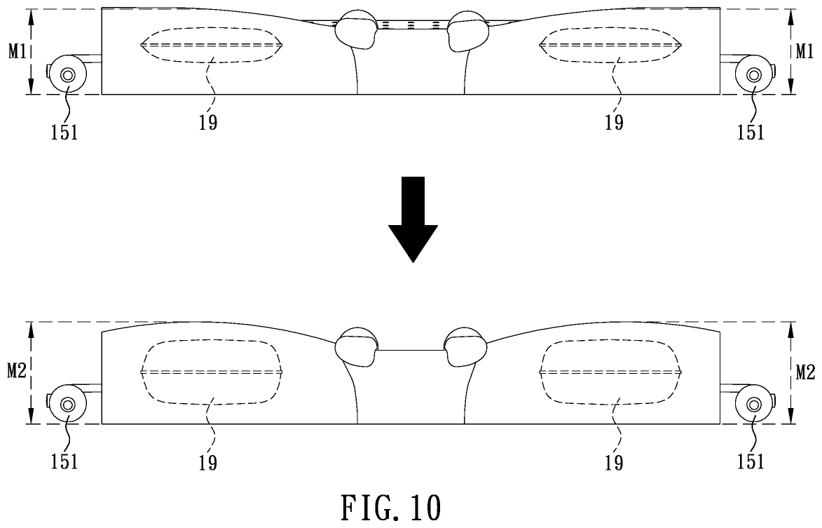

Referring to FIGS. 7, 8 and 10, when the inflation portion 151 inflates at least one of the two second inflatable airbags 19, the second inflatable airbag 19 gradually expands so that areas of the top surfaces of the pillow body 1 and the pillowcase 2 that correspond in position to the second inflatable airbags 19 are gradually moved upward, thereby abutting against and providing proper support for the neck of a user who is laying sideways and on the side of his or her head on the neck support device S, and restoring and maintaining the head, cervical spine and upper thoracic spine alignment of the user. The height of the top surface, or more precisely a point of the top surface that corresponds to the highest point of a second inflatable airbag 19 along the Z axis shown in FIG. 10, of the neck support device S relative to the bottom surface of the neck support device S when the top surface is not displaced by the expansion of the second airbag 19 can be defined as a third height M1. The height of the top surface, or more precisely a point of the top surface that corresponds to the highest point of the second inflatable airbag 19 along the Z axis shown in FIG. 10, of the neck support device S relative to the bottom surface of the neck support device S when the top surface is displaced by the second inflatable airbag 19 expanding to the second maximum expansion state can be defined as a fourth height M2. In certain embodiments, the ratio of the third height M1 to the fourth height M2 can range between 1:1.48 and 1:2.01, and in certain embodiments, between 1:1.64 and 1:1.85. Accordingly, by the design of the second inflatable airbags 19, the height and the firmness of an area of the neck support device S that abuts against and corresponds to the neck of a user when the user is lying sideways on the neck support device S, or more particularly, when the user is lying sideways on the neck support device S sleep and on the side of his or her head, can also be adjusted to keep the neck of the user at a proper position and allow the head, cervical spine and upper thoracic spine of the user to be aligned properly.

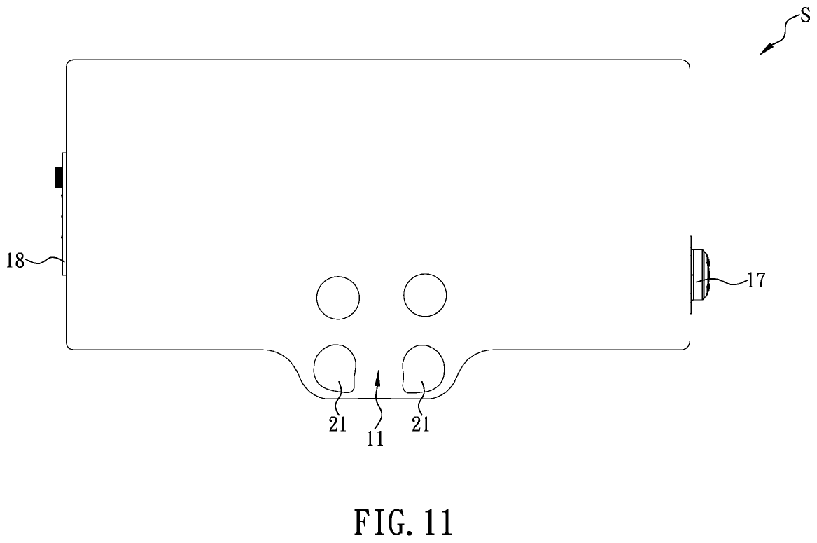

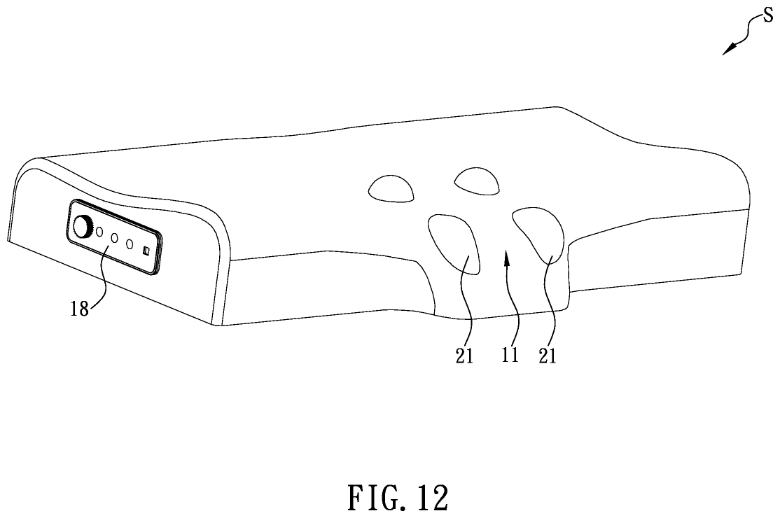

Referring to FIGS. 11 and 12, in certain embodiments, one side of the neck support device S is arranged with an electric inflation device 18. The electric inflation device 18 includes an electric inflation pump, and has at least one pipe body, a first air valve and at least one second air valve that are embedded within and not exposed from the neck support device S. Accordingly, a user can easily inflate or deflate the first inflatable airbag 13 and/or the second inflatable airbags 19 by operating at a control panel of the electric inflation device 18.

When a user uses the neck support device S, in addition to utilizing the inflation effect of the first airbag 13 and the second airbags 19 to support, and restore the alignment of, the head, cervical spine and upper thoracic spine, and stretch the muscles of the neck and shoulder(s), physical therapy such as electrotherapy and/or heat therapy can also at the same time be performed on the user to relax the tight muscles of the neck and shoulders, so as to improve local blood circulation and relieve pain in the head, neck and shoulders, throbbing arm pain and arm numbness.

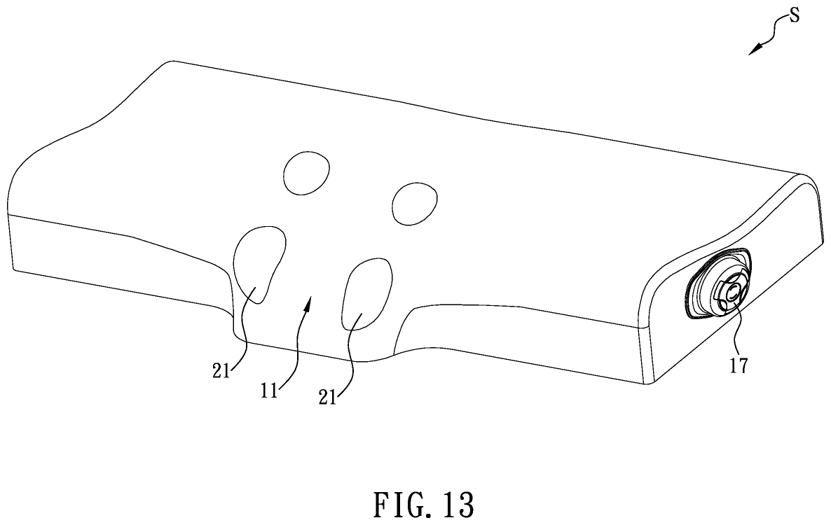

Referring to FIGS. 1-8, the neck support device S can also be provided with a plurality of physical therapy portions 21 that are substantially located at positions at least corresponding to the center support body 11. In certain embodiments, the physical therapy portions 21 can be electrode units that are fastened to positions on the pillowcase 2 that correspond to the upper shoulders and the back of the neck of the user when he or she is laying on the neck support device S in a supine position, so that when the user's head rests on the neck support device S, the physical therapy portions 21 can abut against at least one of the neck and the upper shoulders of the user, allowing the electrode units to stimulate the nerves and muscles of the upper neck, lower neck and upper shoulder junctions. In certain embodiments, the physical therapy portions 21 can be electrode units that are applied with or made of electrical conductive materials and/or sticky gel. The physical therapy portions 21 can be electrically connected to a physical therapy device 17, as shown in FIGS. 11 and 13, to receive electric power transmitted from the physical therapy device 17, so that the physical therapy portions 21 can produce the effects of electrotherapy and/or thermotherapy. In certain embodiments, the physical therapy device 17 is a low-frequency therapy device (also referred to as transcutaneous electrical nerve stimulator, TENS) or an electrical muscle stimulation (EMS) device. At least one button may be arranged on the physical therapy device 17, so that a user can operate on the button to enable the physical therapy device 17 to start outputting and/or stop outputting electrical pulses, and/or outputting electrical pulses of different magnitudes and/or frequencies. In certain embodiments, the physical therapy device 17 is configured to receive wireless signals, such as Bluetooth and/or WiFi signals, so that a user can operate on a wireless controller to control the physical therapy device 17 to start outputting and/or stop outputting electrical pulses, and/or outputting electrical pulses of different magnitudes and/or frequencies. In certain embodiments, at least one of the physical therapy portions 21 and the physical therapy device 17 can be arranged on the pillowcase 2, and can be removed from the pillowcase 2, so that the pillowcase 2 may be washed without damaging any of the physical therapy portions 21 and the physical therapy device 17, while in certain other embodiments, at least one of the physical therapy portions 21 and the physical therapy device 17 may be arranged on the pillow body 1, and the pillowcase 2 may be provided with a through hole through which the physical therapy device 17 can be exposed from the pillowcase 2. However, the present disclosure is not limited thereto.

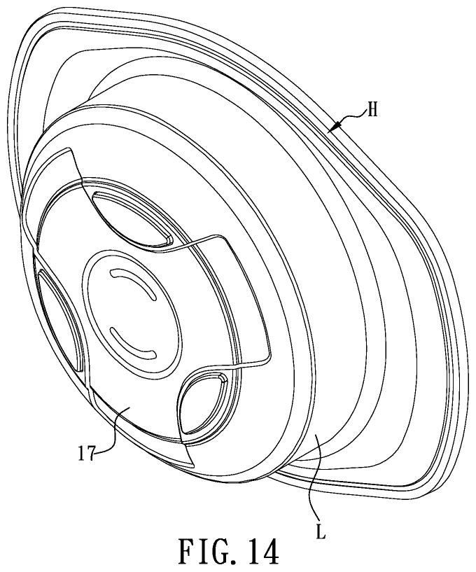

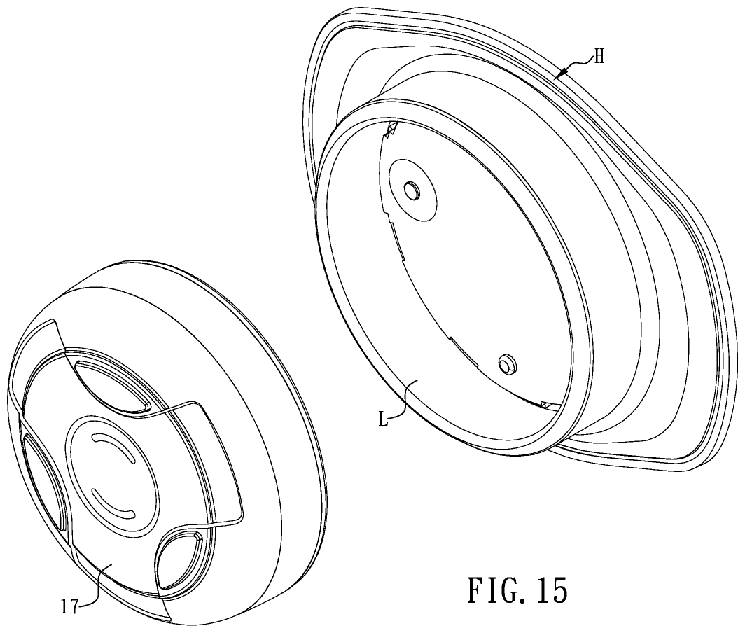

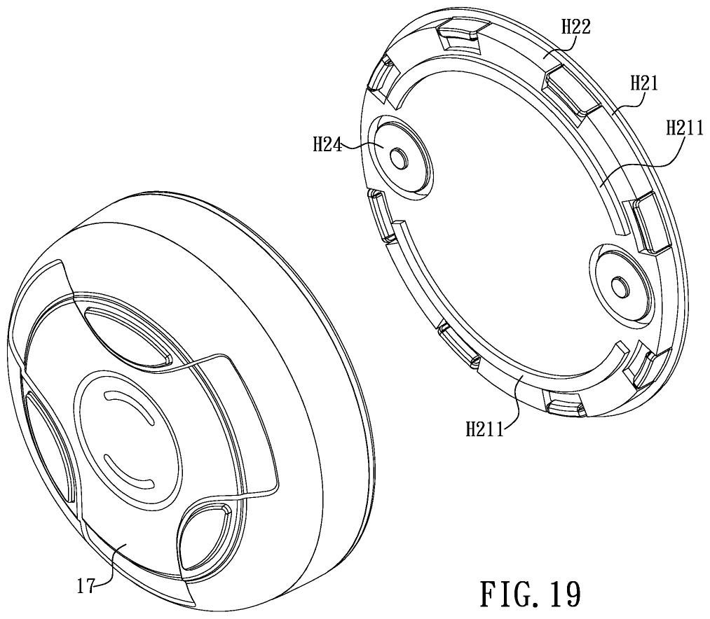

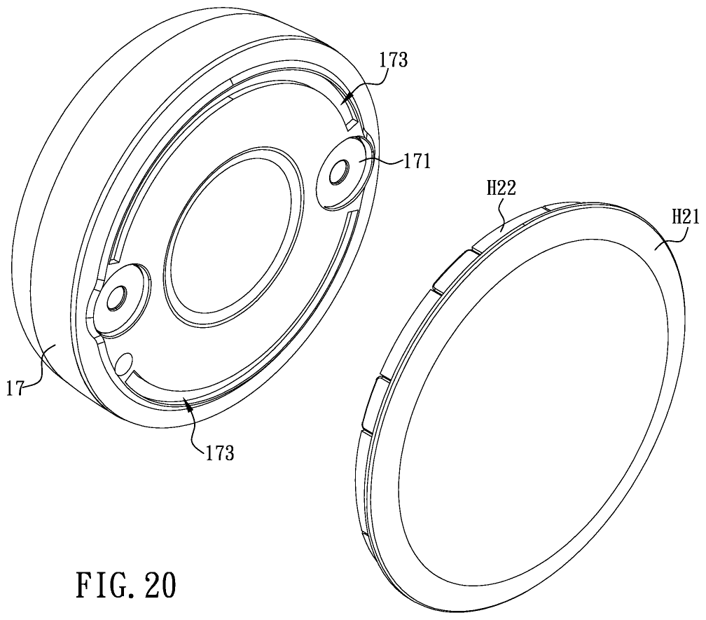

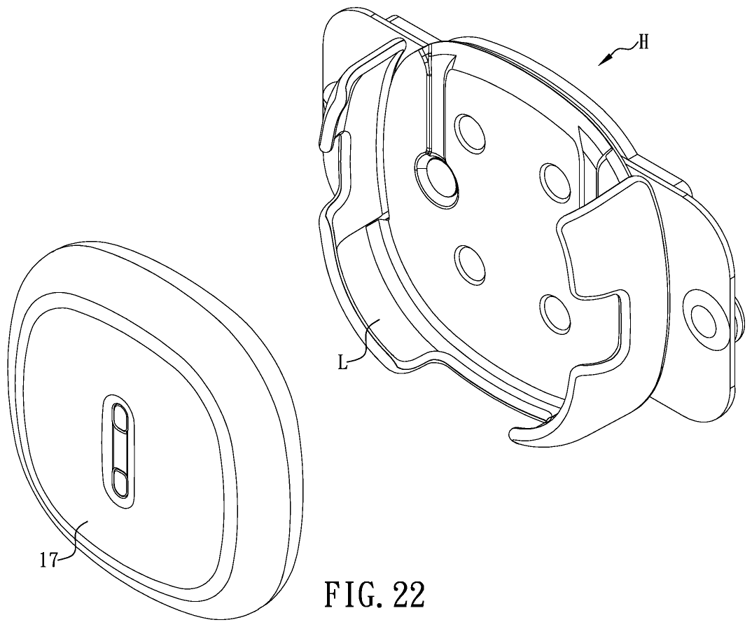

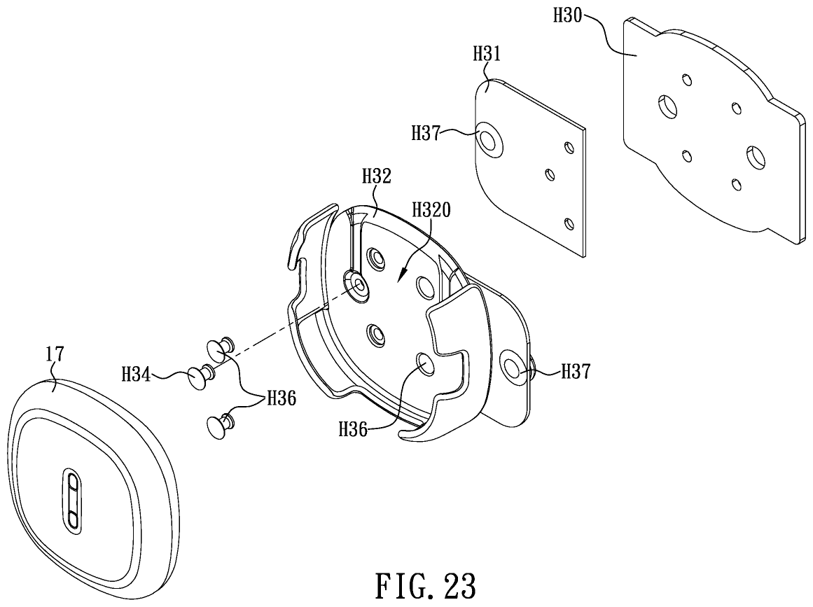

Referring to FIGS. 14-23, the physical therapy device 17 can be held on the neck support device S by a holder H that is designed with a secure releasable locking mechanism L to fix the physical therapy device 17 on, and release the physical therapy device 17 from, that is, the physical therapy device 17 can be removed from, the holder H on demand. Therefore, whenever the neck support device S needs to be washed in water, for example, to wash the pillowcase 2, the physical therapy device 17 can be removed from the holder H to prevent damage to the physical therapy device 17. In certain embodiments, the holder H with the releasable locking mechanism L may be a harness with a releasable lock. The holder H can be provided with at least one conductive member to transmit the electricity produced by the physical therapy device 17 and/or the battery pack thereon/therein to the physical therapy portions 21, so that the physical therapy portions 21 can provide electrotherapy and/or heat therapy.

Referring to FIGS. 14-17, in certain embodiments, the holder H includes a base plate H11, a positioning body H12, an inner plate H13, a plurality of electrical conductive connectors H14, and a plurality of conductive-wire connectors H15. The base plate H11 and the positioning body H12 can be assembled with each other. A rear surface of the base plate H11 can be provided with at least one hook-and-loop fastener, so as to be attached to the neck support device S, for example, to the pillowcase 2 or to the pillow body 1. The positioning body H12 can be made of at least one elastic material, and formed with an accommodating slot H120 on the front surface thereof. The inner plate H13 can be arranged on the bottom surface of the accommodating slot H120. The inner diameter of the accommodating slot H120 can be slightly smaller than the outer diameter of the physical therapy device 17, for example, being smaller by 0.1% to 5% of the outer diameter of the physical therapy device 17, and the slot wall of the accommodating slot H120 can form the releasable locking mechanism, by, when the physical therapy device 17 is placed within the accommodating slot H120, expanding to a small extent, due to the flexibility of the accommodating slot H120, to wrap and abut firmly against the periphery of the physical therapy device 17, so as to position the physical therapy device 17 on the positioning body H12. When the positioned physical therapy device 17 is pulled outward from the positioning body H12 with a force that is greater than a force by friction that is exerted by the slot wall of the accommodating slot H120 (that is, the releasable locking mechanism L) to the physical therapy device 17, the physical therapy device 17 can be removed from the releasable locking mechanism L.

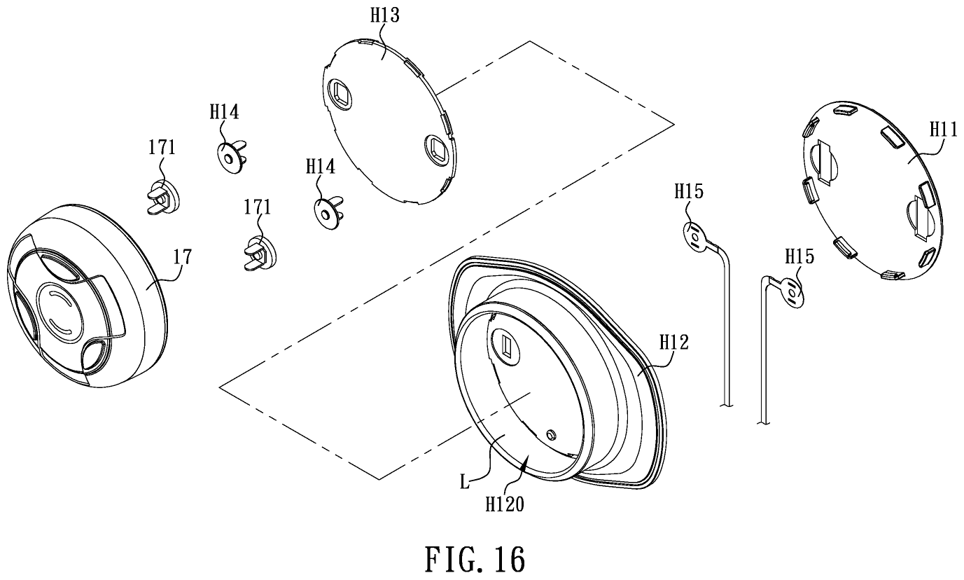

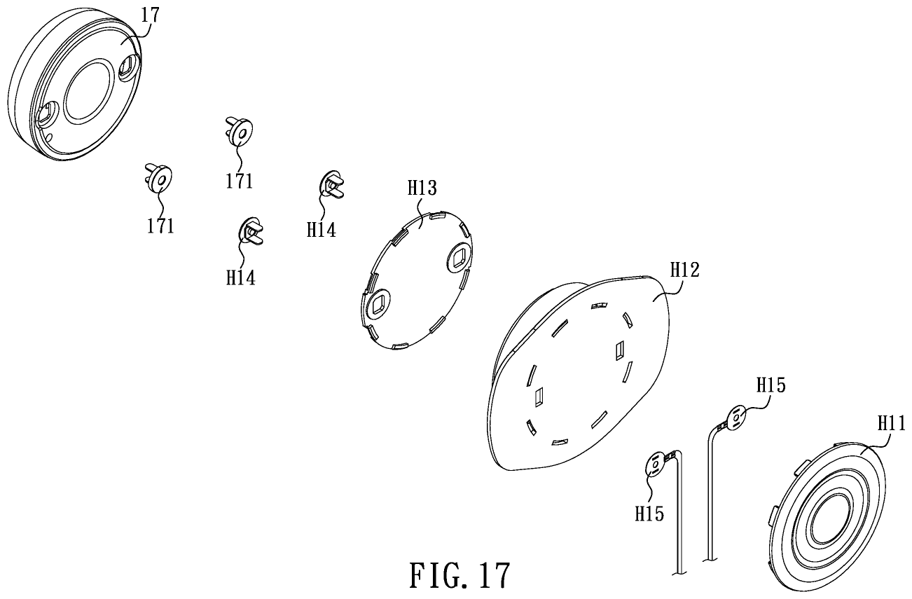

Referring again to FIGS. 16 and 17, the electrical conductive connectors H14 can be placed in the accommodating slot H120, and extend through, in sequence, the inner plate H13 and the bottom surface of the accommodating slot H120 and to the base plate H111. The conductive-wire connectors H15 can be located between the positioning body H12 and the base plate H11, and electrically connected to the corresponding electrical conductive connectors H14 respectively. Each of the conductive-wire connectors H15 can be electrically connected with at least one external conductive wire. Further, the rear side of the physical therapy device 17 can be provided with a plurality of electrical conductive members 171. After the physical therapy device 17 is placed in the accommodating slot H120, each of the electrical conductive members 171 can be electrically connected with a corresponding electrical conductive connectors H14, so that the electric current (current pulse signals) outputted by the physical therapy device 17 can pass in sequence through the electrical conductive member 171 and the electrical conductive connectors H14 and be transmitted to the conductive-wire connector H15. However, in certain embodiments, the electrical conductive connector H14 and the conductive-wire connector H15 may be integrated into one piece, and the electrical conductive connector H14 can be electrically connected with an external conductive wire.

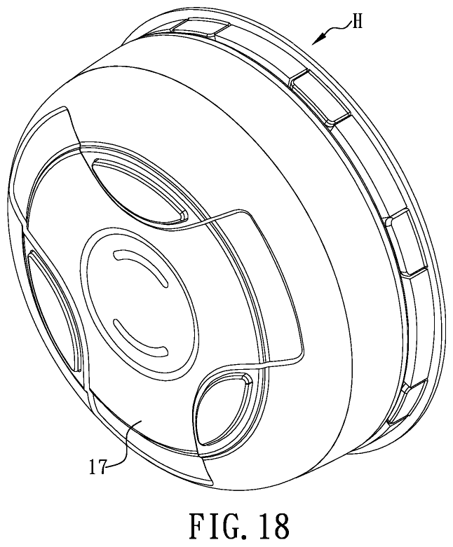

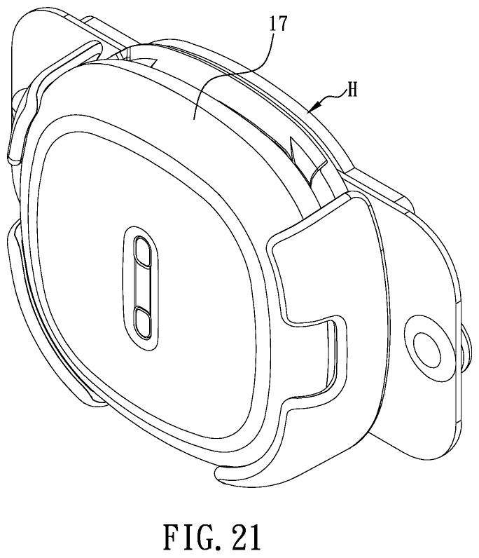

Referring to FIGS. 18-20, in certain embodiments, the holder H may include a base plate H21, a positioning body H22, and a plurality of electrical conductive connectors H24. The base plate H21 and the positioning body H22 can be assembled with each other, and the electrical conductive connectors H24 can be fixed on the positioning body H22. The front surface of the positioning body H22 can be protrudingly formed with at least one protruding rail H211 which serves as the releasable locking mechanism L The rear surface of the physical therapy device 17 can be formed with at least one chamber 173. The width and/or length of the chamber 173 can be slightly smaller than the corresponding width and/or length of the protruding rail H211, for example, being smaller by 0.1% to 5% of the width of the protruding rail H211. When assembling the physical therapy device 17 to the positioning body H, the protruding rail H211 can be inserted into the corresponding chamber 173, and the electrical conductive member(s) 171 can at the same time be electrically connected with the corresponding electrical conductive connector(s) H24. When the positioned physical therapy device 17 is pulled outward from the positioning body H22 with a force that is greater than the clamping force between the protruding rail(s) H211 and the corresponding chamber(s) 173, the physical therapy device 17 can be removed from the releasable locking mechanism L Referring to FIGS. 21-23, in certain embodiments, the holder H may include at least one base plate H31, a positioning body H32, and a plurality of electrical conductive connectors H34. The front side of the base plate H31 can abut against the rear side of the positioning body H32, and the rear side of the base plate H31 can abut against a connection plate H30 (for example, at least one hook-and-loop fastener). The base plate H31, the positioning body H32 and the connection plate H30 can be assembled into one piece through a plurality of fastening members H36. The front side of the positioning body H32 is formed with an accommodating room H320, and the physical therapy device 17 can be placed in the accommodating room H320, with the surrounding wall defining and of the accommodating room H320 forming the releasable locking mechanism L. A part of the base plate H31 that is not blocked by the positioning body H32 when the positioning body H32 and the base plate H31 are assembled can be provided with at least one fixing member H37 (for example, a button). The fixing member H37 can be buckled with a corresponding fixing member on the neck support device S, so that the holder H can be more firmly positioned on the neck support device S.

In other words, referring to FIGS. 14-23, the holder H may be provided with various electrical conductive/connecting members as described supra that enable electrical conduction between the physical therapy device 17 and the physical therapy portion(s) 21 and/or electrodes positioned under the neck and on the upper shoulders. Such a physical therapy device-holder-physical therapy portion set-up enables the use of TENS, EMS, and other electrotherapy/heat therapy devices to treat the neck and upper shoulders of a user, and to relieve his or her headaches, neck pain, shoulder pain and radiating pain down the arms.

Further, when the neck support device S needs to be washed, the releasable locking mechanism L can be unlocked to release and remove the physical therapy device 17 before the neck support device S is washed in water, so as to prevent damage to the water sensitive electronics within the physical therapy device 17. In certain embodiments, the physical therapy device 17 can be disposed directly on the neck support device S without the holder H, and is electrically connected to the physical therapy portions 21 to transmit electricity to the physical therapy portions 21.

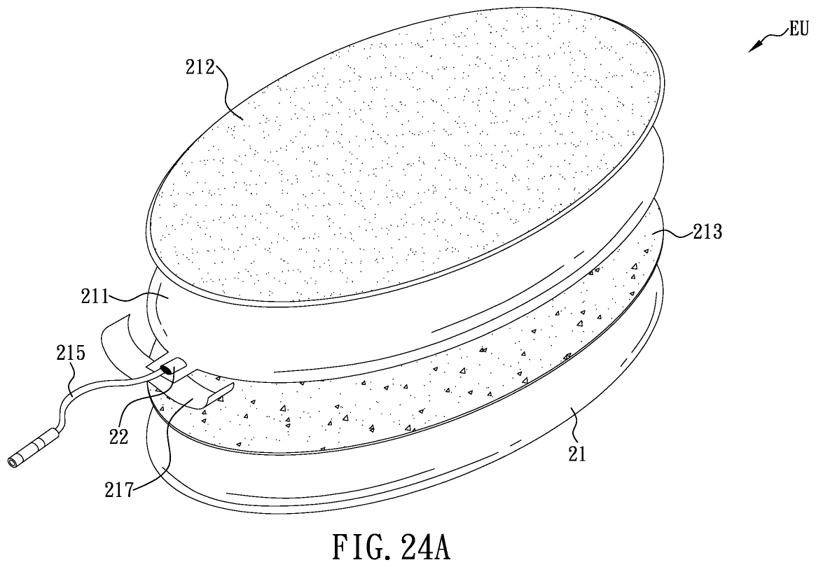

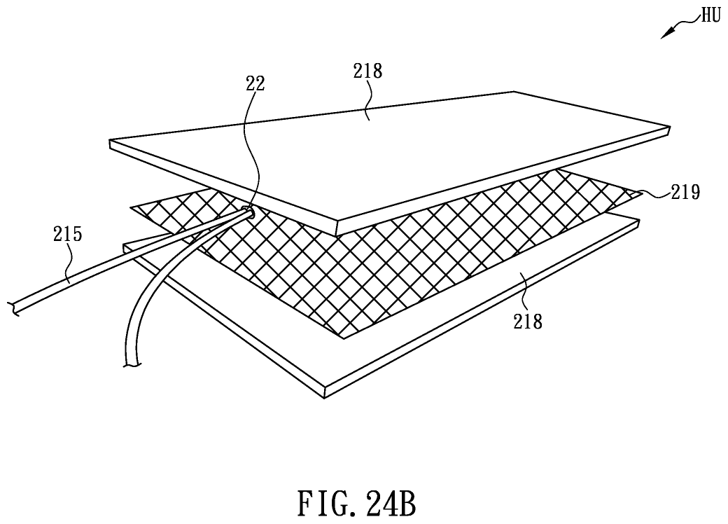

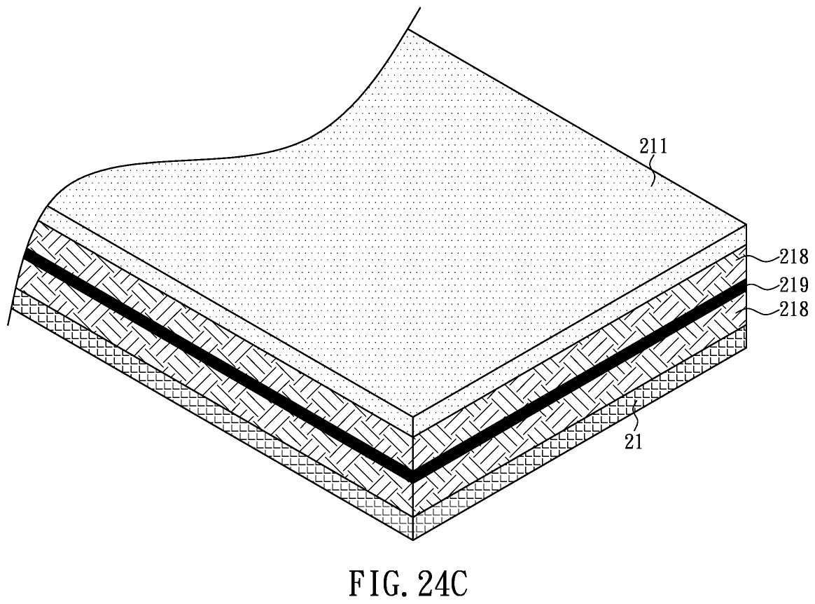

Referring to FIG. 24A, in certain embodiments, a physical therapy portion 21 can be arranged with an electrotherapy unit EU. The electrotherapy unit EU can include a conductive layer 211, optionally a thin film layer 212, and an adhesion layer 213. The conductive layer 211 may be a layer of conductive fibers, conductive film, conductive cloth, aluminum foil, or a mixture thereof, or be made of other conductive materials. One side of the conductive layer 211 can be disposed with the optional thin film layer 212 (for example, a layer of conductive gel), while the other side of the conductive layer 211 can be disposed with the adhesion layer 213. The conductive layer 211 can be electrically connected to a metal member 22, and the metal member 22 can be electrically connected to a conductive wire 215. An insulative band 217 can be wound around and fix the metal member 22 and the conductive wire 215. The adhesion layer 213 (for example, a layer of glue) can be fixed to the pillowcase 2, or in certain embodiments, to the pillow body 1, so that for a user to use the physical therapy portion 21, as long as the conductive layer 211, or if the thin film layer 212 exists, the thin film layer 212, is abutted against his or her skin, and the conductive wire 215 is electrically connected to the physical therapy device 17, electric power from the physical therapy device 17 such as a TENS or EMS device can be received by the physical therapy portion 21, and the physical therapy portion 21 can output electrical stimulation to the neck of a user to achieve the effect of electrotherapy. However, the present disclosure is not limited thereto. In certain embodiments, when the thin film layer 212 is omitted, the conductive layer 211 is in direct contact with the skin. The adhesion layer 213 may include fabric such as nonwoven or cotton fabric, and can be connected with a conductive sheet such as an aluminum foil, so as to be better connected to the pillow body 1 or the pillowcase 2.

Referring to FIG. 24B, in certain embodiments, a physical therapy portion 21 can be arranged with a heat therapy unit HU. The heat therapy unit HU can include two cushion layers 218 (for example, being made of non-woven fabric) and a heat-generating layer 219. The heat-generating layer 219 can be made of metal material (e.g., iron-chromium-aluminum alloy wires, nickel-chromium alloy wires, etc.), graphene, carbon fiber material, or other electrothermal materials, etc., so as to generate heat when electric current passes therethrough. The heat-generating layer 219 can be sandwiched between the two cushion layers 218, and the outer surface of one of the cushion layers 218 can be fixated on the corresponding pillowcase 2, or in certain embodiments, to the pillow body 1. The heat-generating layer 219 can be electrically connected with the metal member 22 to receive electric power from the physical therapy device 17 through the conductive wire 215, so that the physical therapy portion 21 generates heat that is conducted away through the cushion layers 218, and heats the neck of the user to relax blood vessels and increase local blood circulation and the rate of metabolism, such that inflammatory substances are quickly expelled from the human body and self-healing abilities of the muscle tissue can be improved. At the same time, warm compresses can increase soft tissue resilience and reduce muscle spasms, so as to relieve one's pain and relax one's emotions. In certain embodiments, the cushion layers 218 may be omitted, and the heat-generating layer 219 is in direct contact with the skin.

Referring to FIG. 24C, in certain embodiments, a physical therapy portion 21 can be arranged with a combined electrotherapy-heat therapy unit. The combined electrotherapy-heat therapy unit can be formed by stacking the foregoing electrotherapy unit EU and heat therapy unit HU. For example, a heat-generating layer 219 is provided between two cushion layers 218, and the outer surface of one of the cushion layers 218 can be covered with the conductive layer 211, such as a layer of conductive fiber. The heat-generating layer 219 and the conductive layer 211 can receive electric power from the physical therapy device 17 through the same or different metal members 22. The conductive layer 211 can output electrical stimulation to the neck of the user, while the heat-generating layer 219 can generate heat which passes through the cushion layers 218 and the conductive layer 211 to heat the neck of the user.

However, the present disclosure is not limited thereto. As long as the structure of a physical therapy portion 21 can achieve the result and effects of physical therapy, such a structure is within the definition of the physical therapy portion 21 according to the present disclosure.

Referring to FIG. 8, the bottom plate 16 can be assembled to the bottom surface of the pillow body 1 to cover the first chamber 110 and the second chambers 190, and to prevent the first inflatable airbag 13, the second inflatable airbags 19 and the pipe bodies 152 from being separated from the pillow body 1. However, in other embodiments, the bottom plate 16 may be omitted, and the first inflatable airbag 13, the second inflatable airbags 19 and the pipe bodies 152 may be held by the elasticity of the pillow body 1 or be clamped and fixed by other clamping mechanism.

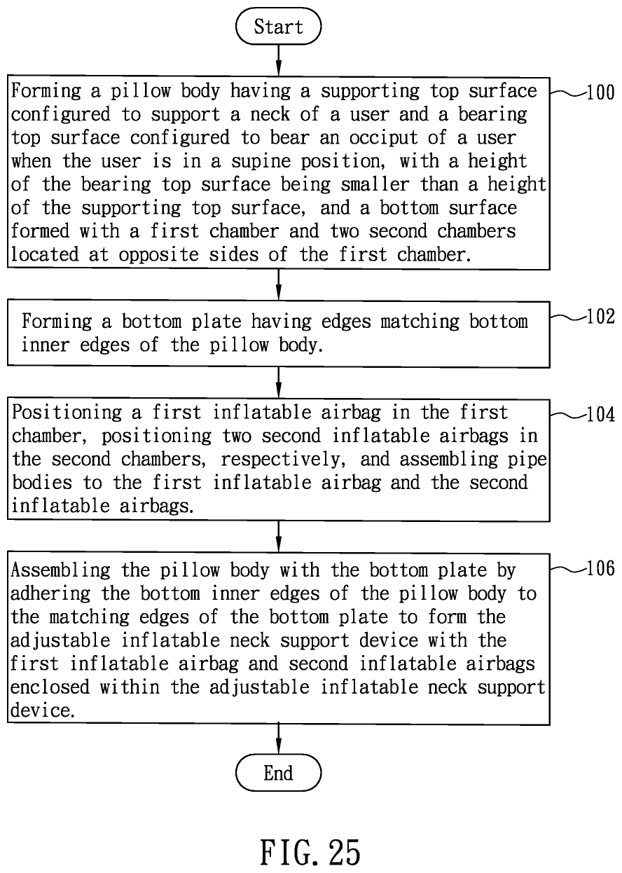

Another aspect of the present disclosure is directed to a method for manufacturing the adjustable inflatable neck support device S. It is noted that the method is not limited to the sequence of the procedures as described either infra or in FIG. 25, and the sequence may vary according practical requirements and/or user preferences without departing from the spirit and scope thereof.

Referring to FIG. 25 in view of FIGS. 1, 2, 7 and 8, step 100 includes forming the pillow body 1 having a supporting top surface configured to support a neck of a user, a bearing top surface configured to bear an occiput of a user when the user is in a supine position, with a height of the bearing top surface being smaller than a height of the supporting top surface, and a bottom surface formed with the first chamber 110 and the two second chambers 190 located at opposite sides of the first chamber 110. In certain embodiments, the pillow body 1 can be formed via a molding process, such as compression molding, injection molding, centrifugal molding, extrusion molding, reaction injection molding, thermoforming, etc. Step 102 includes forming a bottom plate 16 having edges corresponding to and matching the bottom inner edges of the pillow body 1 and configured to cover the first chamber 110 and the two second chambers 190. In certain embodiments, the bottom plate 16 can be formed via a molding process, such as compression molding, injection molding, centrifugal molding, extrusion molding, reaction injection molding, thermoforming, etc. The bottom plate 16 can be a portion separated and independent from the pillow body 1, or pivotally connected to the pillow body 1 to collectively form a shell shaped structure.

Step 104 includes positioning the first inflatable airbag 13 in the first chamber 110, positioning the two second inflatable airbags 19 in the second chambers 190, respectively, extending pipe bodies 152 through pipe holes formed on pillow body 1 and through pipe cavities communicating with the pipe holes and formed in the pillow body 1, and assembling the pipe bodies 152 to the first inflatable airbag 13 and the second inflatable airbags 19.

Step 106 includes assembling the pillow body 1 with the bottom plate 16 by adhering the bottom inner edges of the pillow body 1 to the matching edges of the bottom plate 16 to form the adjustable inflatable neck support device S with the first inflatable airbag 13 and the second inflatable airbags 19 enclosed within the adjustable inflatable neck support device S. In certain embodiments, to ensure proper adhesion strength in keeping pillow body 1 and the bottom plate 16 together for longtime use, the adhering can be via glue, adhesive tapes, heat combined with compression, and/or other fastening methods applying foam or other material, along the entire peripheral edges of the bottom plate 16 and the entire inner edges of the pillow body 1 the formed adjustable inflatable neck support device S, so as to hold the neck support device S as an enclosed object.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An adjustable inflatable neck support device, comprising:
a center support body having a top surface configured to support a neck of a user and a bottom surface concavely provided with a first chamber;
a first inflatable airbag configured to be accommodated within the first chamber, expand or contract along a displacement axis, and displace the top surface of the center support body along the displacement axis;
two second inflatable airbags, each configured to be accommodated within a corresponding one of second chambers that are concavely provided on the bottom surface of the neck support device and located at opposite sides of the first chamber, expand or contract along the displacement axis, and displace a top surface of the neck support device along the displacement axis; and
an inflation device configured to be connected to the first inflatable airbag and the second inflatable airbags, respectively, and inflate or deflate the first inflatable airbag and the second inflatable airbags to change expansion or contraction degrees of the first inflatable airbag and the second inflatable airbags,
wherein the neck support device is divided into at least a supporting portion including the center support body and the first inflatable airbag and a bearing portion configured to bear an occiput of a user when the user is in a supine position, and a height of a top surface of the bearing portion is lower than a height of a top surface of the supporting portion.

2. The neck support device according to claim 1, wherein the inflation device comprises:
a plurality of pipe bodies;
a first air valve configured to be assembled to a first one of the pipe bodies;
at least one second air valve configured to be assembled to a second one of the pipe bodies; and
at least one inflation portion configured to be connected to the first inflatable airbag and the second inflatable airbags through the pipe bodies, inflate or deflate the first inflatable airbag when the first air valve is opened, and inflate or deflate the second inflatable airbags when the second air valve is opened.

3. The neck support device according to claim 1, wherein the first inflatable airbag is configured to expand to a first maximum expansion state with a maximum height of the first inflatable airbag in a direction of the displacement axis being 5.5 cm to 9.5 cm.

4. The neck support device according to claim 1, wherein at least one of the second inflatable airbags is configured to expand to a second maximum expansion state with a maximum height of the second inflatable airbag in a direction of the displacement axis being 6.5 cm to 10.5 cm.

5. The neck support device according to claim 1, further comprising a pillowcase and a plurality of physical therapy portions fastened to the pillowcase, wherein at least one of the physical therapy portions is fastened to a position on the pillowcase that corresponds to a position of the center support body, and is configured to abut against the neck of the user and be electrically connected to a physical therapy device to receive electric power transmitted from the physical therapy device.

6. The neck support device according to claim 5, wherein at least one of the physical therapy portions is arranged with an electrotherapy unit configured to receive the electric power transmitted from the physical therapy device and output electrical stimulation to the neck of the user.

7. The neck support device according to claim 5, wherein at least one of the physical therapy portions is arranged with a heat therapy unit configured to receive the electric power transmitted from the physical therapy device and generate heat to heat the neck of the user.

8. The neck support device according to claim 5, wherein at least one of the physical therapy portions is arranged with a combined electrotherapy-heat therapy unit configured to receive the electric power transmitted from the physical therapy device, output electrical stimulation to the neck of the user and generate heat to heat the neck.

9. The neck support device according to claim 1, comprising a pillow body and a pillowcase, wherein the pillowcase is configured to cover the pillow body, the first inflatable airbag and the second inflatable airbags are arranged within the pillow body, the inflation device is external to the pillow body and the pillowcase, and the pipe bodies are configured to extend into the pillow body through the pillowcase.

10. The neck support device according to claim 1, wherein each of the first and second inflatable airbags has an expandable top end and a non-expandable base.

11. The neck support device according to claim 1, wherein the first inflatable airbag has a rounded first end configured to face an upper inner surface of the first chamber, and an opposite second end that is a flat base configured to face a bottom opening of the first chamber, and at least one of the second inflatable airbags has a flat top end configured to face an upper inner surface of the second chamber and a flat bottom base opposite to the flat top end and configured to face a bottom opening of the second chamber.

12. The neck support device according to claim 1, wherein each of the second chambers is larger than the first chamber, and each of the second inflatable airbags under an uninflated state is greater in at least one of length and volume than the first inflatable airbag under an uninflated state.

13. The neck support device according to claim 1, wherein the center support body is firmer than a center portion of the bearing portion that corresponds to a back of the head of the user when the user lays his or her neck on the center support body.

14. The neck support device according to claim 1, wherein side portions of the pillow body that include corresponding portions of the supporting portion and the bearing portion and correspond to the second chambers are firmer than a rest of the bearing portion.

15. A method for manufacturing an adjustable inflatable neck support device, comprising:
forming a pillow body having a supporting top surface configured to support a neck of a user, a bearing top surface configured to bear an occiput of a user when the user is in a supine position, and a bottom surface formed with a first chamber and two second chambers located at opposite sides of the first chamber, wherein a height of the bearing top surface is smaller than a height of the supporting top surface;
forming a bottom plate having edges matching bottom inner edges of the pillow body;
positioning a first inflatable airbag in the first chamber, positioning two second inflatable airbags in the second chambers, respectively, and assembling pipe bodies to the first inflatable airbag and the second inflatable airbags; and
assembling the pillow body with the bottom plate by adhering the bottom inner edges of the pillow body to the matching edges of the bottom plate to form the adjustable inflatable neck support device with the first inflatable airbag and the second inflatable airbags enclosed within the adjustable inflatable neck support device.
